(19) United States
(12) Patent Application Publication
(10) Pub. No.: US 2024/0117124 A1
(43) Pub. Date: Apr. 11, 2024

(54) MODIFIED SILICONE RUBBER AND PREPARATION METHODS THEREOF

(71) Applicant: WUHAN UNITED IMAGING HEALTHCARE CO., LTD., Wuhan (CN)

(72) Inventors: Cong LIU, Wuhan (CN); Kang SI, Shanghai (CN); Yu LIU, Wuhan (CN); Zhifang LI, Wuhan (CN)

(73) Assignee: WUHAN UNITED IMAGING HEALTHCARE CO., LTD., Wuhan (CN)

(21) Appl. No.: 18/322,575

(22) Filed: May 23, 2023

Related U.S. Application Data
(63) Continuation of application No. PCT/CN2022/143172, filed on Dec. 29, 2022.

(30) Foreign Application Priority Data
Sep. 21, 2022 (CN) 202211152905.1

(51) Int. Cl. C08G 77/20; A61B 8/00; C08F 20/44; C08F 36/06; C08G 77/08; C08G 77/12; C08G 77/38

(52) U.S. Cl. CPC C08G 77/20; A61B 8/4281; C08F 20/44; C08F 36/06; C08G 77/08; C08G 77/12; C08G 77/38

(57) ABSTRACT

Modified silicone rubber and a preparation method thereof are provided in the present disclosure. A composition of raw materials for preparing the modified silicone rubber may include a raw material that can form silicone rubber and a raw material that can form a modified material. The raw material that can form the silicone rubber may include a first component and a second component. The raw material that can form the modified material may include a third component and a fourth component. The preparation method of the modified silicone rubber may include mixing the composition of the raw materials for preparing the modified silicone rubber and obtaining the modified silicone rubber by curing and molding the mixture.

### CROSS REFERENCE TO RELATED APPLICATIONS

This specification is a Continuation of International Application No. PCT/CN2022/143172 filed on Dec. 29, 2022, which claims priority of the Chinese Patent Application No. 202211152905.1, filed on Sep. 21, 2022, the entire contents of each of which are hereby incorporated by reference.

### TECHNICAL FIELD

The present disclosure relates to the technical field of acoustic materials, and in particular to modified silicone rubber and preparation methods thereof.

### BACKGROUND

Ultrasonic diagnostic equipment is a clinical diagnostic equipment that applies ultrasound in acoustics to medical disciplines. It has the advantages of non-invasiveness, high identification of soft tissues, and no radiation damage to the human body. Its working principle is to generate incident ultrasonic waves (also referred to as transmitted waves) and receive reflected ultrasonic waves (also referred to as echoes) through the ultrasonic probe, and finally display them on the oscilloscope in the form of echoes. The ultrasonic probe is a key component to realize the conversion between ultrasonic signals and electrical signals in the ultrasonic diagnostic equipment. The ultrasonic probe is mainly composed of an acoustic lens, a matching layer, a piezoelectric element, a back, and other components. The acoustic lens is located on the outermost layer of the ultrasonic probe and is in direct contact with the human body medium. The acoustic lens as a contact interface not only needs to have as low acoustic attenuation as possible, but also needs to have an acoustic impedance similar to that of human tissue, to achieve the impedance matching effect with human tissue, thereby improving the sensitivity of the ultrasonic probe, and accordingly, improving the image quality. At present, it is difficult for the sound-transparent materials used for the preparation of acoustic lenses to have relatively low sound attenuation, relatively high acoustic impedance matching effect and hardness simultaneously, so it is difficult to meet the requirements of relatively high imaging sensitivity and imaging quality, and relatively long service life. Therefore, it is desirable to provide modified silicone rubber and a preparation method thereof, to obtain a sound-transparent material meeting the above requirements simultaneously.

### SUMMARY

One of the embodiments of the present disclosure provides a composition of raw materials for preparing modified silicone rubber. The composition may include a raw material that can form silicone rubber and includes a first component and a second component. The composition may further include a raw material that can form a modified material and includes a third component and a fourth component.

In some embodiments, a mass fraction of the first component may be in a range of 50-150. A mass fraction of the second component may be in a range of 0-20 and greater than 0. A mass fraction of the third component may be in a range of 25-150. A mass fraction of the fourth component may be in a range of 0-20 and greater than 0.

In some embodiments, a mass ratio of the first component, the second component, the third component, and the fourth component may be 10:1:(5-10):(0.5-1).

In some embodiments, the first component may include vinyl silicone rubber and a cross-linking agent. The second component may include a catalyst capable of catalyzing an addition reaction of the vinyl silicone rubber and the cross-linking agent.

In some embodiments, a structural formula of the vinyl silicone rubber may be shown in formula I:

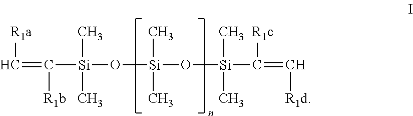

R₁a, R₁b, R₁c, and R₁d may be independently selected from H, substituted or unsubstituted C₁-C₅ straight chain or branched chain alkyl, substituted or unsubstituted C₆-C₂₀ aryl, n≥1000. The cross-linking agent may include a Si—H bond. The second component may include at least one of transition metals of Group VIII of a periodic table, a compound thereof, or a complex thereof.

In some embodiments, the second component may further include at least one of methyl silicone oil, vinyl silicone oil, hydroxyl silicone oil, hydroxymethyl fluoro silicone oil, or epoxy-terminated silicone oil.

In some embodiments, a structural formula of the vinyl silicone rubber may be shown in formula I′:

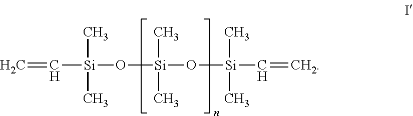

In some embodiments, the cross-linking agent may be polymethylhydrogensiloxane.

In some embodiments, the first component may further include at least one of an inhibitor or a filler.

In some embodiments, the inhibitor may include at least one of an alkynol compound, a nitrogen-containing compound, or organic peroxide.

In some embodiments, the filler may include at least one of white carbon black, titanium dioxide, quartz powder, aluminum oxide, zinc oxide, or tungsten oxide.

In some embodiments, the third component may include a butadiene compound and an acrylonitrile compound, and the fourth component may include a compound capable of catalyzing an addition reaction of the butadiene compound and the acrylonitrile compound. In some embodiments, the third component may include a butadiene compound, and the fourth component includes a catalyst capable of catalyzing an addition reaction of the butadiene compound. In some embodiments, the third component may include a fluorine-containing carbon chain, and the fourth component may include polycarbodiimide.

In some embodiments, a structural formula of the butadiene compound may be shown in formula II,

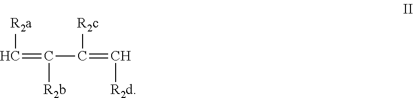

R₂a, R₂b, R₂c, and R₂d may be independently selected from H, substituted or unsubstituted C₁-C₃ straight chain or branched chain alkyl, substituted or unsubstituted C₆-C₂₀ aryl. A structural formula of the acrylonitrile compound may be shown in formula III,

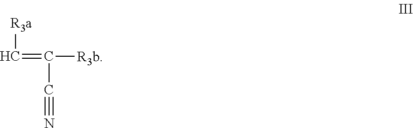

R₃a and R₃b may be independently selected from H, substituted or unsubstituted C₁-C₃ straight chain or branched chain alkyl, substituted or unsubstituted C₆-C₂₀ aryl. A structural formula of the fluorine-containing carbon chain may be shown in formula IV,

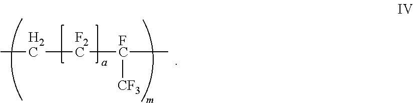

m may not be less than 200, a may be in a range of 1-8, and a may be an integer. The fourth component may include at least one of transition metals of Group VIII of a periodic table, a compound thereof, or a complex thereof.

In some embodiments, the fourth component may include platinum, a platinum-containing compound, or a platinum-containing complex.

In some embodiments, the fourth component may include organotin or organobismuth.

In some embodiments, the butadiene compound may be 1,3-butadiene. The acrylonitrile compound may be acrylonitrile.

The embodiments of the present disclosure further provide a preparation method of modified silicone rubber. The preparation method may include mixing the composition of raw materials for preparing the modified silicone rubber and obtaining the modified silicone rubber by curing and molding the mixture.

In some embodiments, an acoustic impedance of the modified silicone rubber may be within a range of 1.25 Mrayl-1.50 Mrayl. A sound attenuation coefficient of the modified silicone rubber at a frequency of 5 MHz is not greater than 42 dB/cm. A Shore hardness of the modified silicone rubber is in a range of 30 HA-70 HA.

In some embodiments, the modified silicone rubber may be used as a sound-transparent material.

In some embodiments, the sound-transparent material may include a sound-transparent element applied to an ultrasonic probe.

The embodiments of the present disclosure further provide modified silicone rubber prepared by the above preparation method of the modified silicone rubber.

The embodiments of the present disclosure further provide a modified material of silicone rubber. The modified material may be at least one of nitrile rubber, cis-polybutadiene, or fluorine rubber. A raw material forming the modified material may include a third component and a fourth component. The third component may include a butadiene compound and an acrylonitrile compound, and the fourth component may include a catalyst capable of catalyzing an addition reaction of the butadiene compound and the acrylonitrile compound; or the third component may include a butadiene compound, and the fourth component may include a catalyst capable of catalyzing an addition reaction of the butadiene compound; or the third component may include a fluorine-containing carbon chain, and the fourth component may include polycarbodiimide.

In some embodiments, a structural formula of the butadiene compound may be shown in formula II,

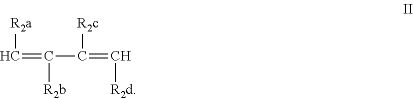

R₂a, R₂b, R₂c, and R₂d may be independently selected from H, substituted or unsubstituted C₁-C₃ straight chain or branched chain alkyl, substituted or unsubstituted C₆-C₂₀ aryl. A structural formula of the acrylonitrile compound may be shown in formula III,

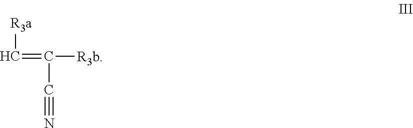

R₃a and R₃b may be independently selected from H, substituted or unsubstituted C₁-C₃ straight chain or branched chain alkyl, substituted or unsubstituted C₆-C₂₀ aryl. A structural formula of the fluorine-containing carbon chain may be shown in formula IV,

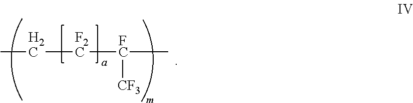

m may not be less than 200, a may be in a range of 1-8, and a may be an integer. The fourth component may include at least one transition metal of Group VIII of a periodic table, a compound thereof, or a complex thereof.

In some embodiments, the fourth component may include platinum, a platinum-containing compound, or a platinum-containing complex.

In some embodiments, the fourth component may include organotin or organobismuth.

In some embodiments, the butadiene compound may 1,3-butadiene. The acrylonitrile compound may be acrylonitrile.

### DETAILED DESCRIPTION

As indicated in the disclosure and claims, the terms “a,” “an,” “an,” and/or “the” are not specific to the singular form and may include the plural form unless the context clearly indicates an exception. Generally speaking, the terms “comprising” and “including” only suggest the inclusion of clearly identified steps and elements, and these steps and elements do not constitute an exclusive list, and the method or device may also contain other steps or elements.

The embodiments of the present disclosure provide a composition of raw materials for preparing modified silicone rubber. The composition may include a raw material that can form silicone rubber and a raw material that can form a modified material. The modified material may be used to prepare the modified silicone rubber (e.g., room temperature vulcanized (RTV) silicone rubber) by modifying the silicone rubber, thereby improving the acoustic impedance of the modified silicone rubber and making the modified silicone rubber have a relatively high acoustic impedance matching effect, relatively low sound attenuation, and relatively high mechanical properties (e.g., hardness).

In some embodiments, the raw material that can form the silicone rubber may include a first component and a second component. In some embodiments, the first component may include vinyl silicone rubber and a cross-linking agent.

In some embodiments, a structural formula of the vinyl silicone rubber may be shown in Formula I:

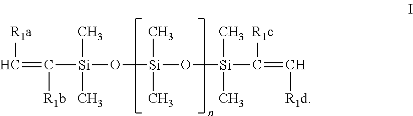

R₁a, R₁b, R₁c, and R₁d may be independently selected from H, substituted or unsubstituted C₁-C₅ straight chain or branched chain alkyl, substituted or unsubstituted C₆-C₂₀ aryl, n≥1000.

In some embodiments, in the structure shown in Formula I, one or more of R₁a, R₁b, R₁c, and R₁d may be selected from H. In the structure shown in formula I, when R₁a, R₁b, R₁c, and R₁d are selected from H simultaneously, the structural formula of the vinyl silicone rubber may be shown in formula I′:

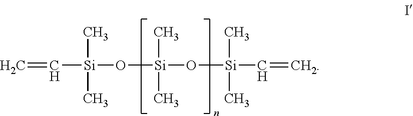

In some embodiments, in the structure shown in Formula I, n may be in a range of 1000-5000. In some embodiments, in the structure shown in Formula I, n may be in a range of 2000-5000. In some embodiments, in the structure shown in Formula I, n may be in a range of 2000-4000. In some embodiments, in the structure shown in Formula I, n may be in a range of 2000-3000. In some embodiments, in the structure shown in Formula I, n may be 1000, 2000, 3000, 4000, or 5000.

In some embodiments, a number average molecular weight of the vinyl silicone rubber may be in a range of 1,000-200,000. In some embodiments, the number average molecular weight of the vinyl silicone rubber may be in a range of 5,000-200,000. In some embodiments, the number average molecular weight of the vinyl silicone rubber may be in a range of 10,000-190,000. In some embodiments, the number average molecular weight of the vinyl silicone rubber may be in a range of 20,000-180,000. In some embodiments, the number average molecular weight of the vinyl silicone rubber may be in a range of 30,000-170,000. In some embodiments, the number average molecular weight of the vinyl silicone rubber may be in a range of 40,000-160,000. In some embodiments, the number average molecular weight of the vinyl silicone rubber may be in a range of 50,000-150,000. In some embodiments, the number average molecular weight of the vinyl silicone rubber may be in a range of 60,000-140,000. In some embodiments, the number average molecular weight of the vinyl silicone rubber may be in a range of 70,000-130,000. In some embodiments, the number average molecular weight of the vinyl silicone rubber may be in a range of 80,000-120,000. In some embodiments, the number average molecular weight of the vinyl silicone rubber may be in a range of 90,000-110,000.

In some embodiments, the cross-linking agent may include a Si—H bond. In some embodiments, a structure of the cross-linking agent may be expressed as R≡SiH, wherein R may be selected from substituted or unsubstituted alkyl or aryl. In some embodiments, the cross-linking agent may be polymethylhydrogensiloxane. In some embodiments, a structural formula of polymethylhydrogensiloxane may be

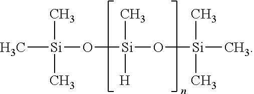

In some embodiments, the first component may further include an additive. For example, the additive may include at least one of an inhibitor or a filler. In some embodiments, the additive may have no effect on a main structure of the silicone rubber.

In some embodiments, the inhibitor may be a reagent capable of inhibiting an addition reaction between the vinyl silicone rubber and the cross-linking agent in the first component. In some embodiments, the inhibitor may include at least one of an alkynol compound, a nitrogen-containing compound, or organic peroxide. For example, the inhibitor may include methyl butynol. As another example, the inhibitor may include 2-methyl-3-butyn-2-ol.

The filler may not only increase the acoustic impedance of the modified silicone rubber by increasing a density of the silicone rubber, but also improve the mechanical properties and wear resistance (e.g., hardness) of the modified silicone rubber. In some embodiments, the filler may include at least one of white carbon black, titanium dioxide, quartz powder, aluminum oxide, zinc oxide, or tungsten oxide. For example, the filler may be the white carbon black.

In some embodiments, the second component may include a catalyst capable of catalyzing a reaction (e.g., an addition reaction) of the vinyl silicone rubber and the cross-linking agent. For example, the second component may have addition catalytic effect on —SiCH═CH₂ in the first component. As another example, the second component may have addition catalytic effect on R≡SiH in the first component. In some embodiments, the second component may include at least one of transition metals of Group VIII of a periodic table, a compound thereof, or a complex thereof. For example, the second component may include platinum, a platinum-containing compound, or a platinum-containing complex. In some embodiments, the compound or the complex of the transition metals of Group VIII of the periodic table in the second component may be used as a catalyst or a curing agent instead of the filler. Accordingly, an amount of the compound or the complex of the transition metals of group VIII of the periodic table in the second compound may be a catalytic amount, while an amount of the added filler may be generally large, even exceed the amount of the silicone rubber. In some embodiments, the compound or the complex of the transition metals of Group VIII of the periodic table in the second component may be a liquid, while the filler may generally exist in a powder form.

In some embodiments, the second component may further include at least one of methyl silicone oil, vinyl silicone oil, hydroxyl silicone oil, hydroxymethyl fluoro silicone oil, or epoxy-terminated silicone oil. For example, the second component may include the vinyl silicone oil.

In some embodiments, the raw material that can form the silicone rubber may be a raw material forming two-component addition type liquid silicone rubber. In some embodiments, the two-component addition type may mean that two components can form the silicone rubber through an addition reaction.

In some embodiments, in the raw material that can form the silicone rubber, the silicone rubber may be the two-component addition type liquid silicone rubber. Merely by way of example, the silicone rubber may be RTV630 silicone rubber or RTV615 silicone rubber produced by Momentive. The RTV630 silicone rubber or the RTV615 silicone rubber may be two-component RTV liquid silicone rubber.

The two-component addition type liquid silicone rubber may usually use polymethylhydrogensiloxane containing a silicon-hydrogen (Si—H) bond as a cross-linking agent, and cure at a room temperature or a high temperature (e.g., 30° C.-100° C., or 50° C.-70° C.) under the action of a catalyst e.g., a platinum catalyst). A main structure of the two-component addition type liquid silicone rubber may be polydimethylsiloxane containing two or more vinyl. The polydiorganosiloxane containing two vinyl may be represented as a structure shown in Formula I′:

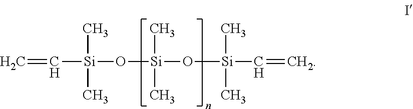

When the first component includes the vinyl silicone rubber and the cross-linking agent that includes the Si—H bond, and the second component includes a catalyst capable of catalyzing a reaction (e.g., an addition reaction) of the vinyl silicone rubber and the cross-linking agent, a curing mechanism of the silicone rubber may be described as the addition reaction of the vinyl silicone rubber and the cross-linking agent including the Si—H bond occur under the catalysis of a catalyst (e.g., a platinum-containing compound). During use, the first component and the second component may be fully mixed in a preset ratio, then placed at the room temperature or high temperature (e.g., 30° C.-100° C., or 50° C.-70° C.) for a preset time (e.g., 12 h-60 h, or 36 h-60 h, or 48 h), and cured to obtain the silicone rubber. For example, the first component and the second component may be fully mixed in the preset ratio, then placed at the room temperature (e.g., 20° C.-30° C.) for 24 h-48 h, and cured to obtain the silicone rubber. As another example, the first component and the second component may be fully mixed in the preset ratio, then placed at the high temperature (e.g., greater than 30° C. and not greater than 100° C., or 50° C.-70° C.) for 12 h-24 h, and cured to obtain the silicone rubber.

The mass fraction may refer to the counting of different components of a mixture in units of mass. The mass fraction may be used to express a mass relationship of the different components in the mixture. The same mass fraction may represent a same mass. In some embodiments, the mass fraction of the first component may be in a range of 50-150. In some embodiments, the mass fraction of the first component may be in a range of 60-140. In some embodiments, the mass fraction of the first component may be in a range of 70-130. In some embodiments, the mass fraction of the first component may be in a range of 75-125. In some embodiments, the mass fraction of the first component may be in a range of 80-120. In some embodiments, the mass fraction of the first component may be in a range of 90-110. In some embodiments, the mass fraction of the first component may be 50, 70, 75, 80, 90, 100, 110, 120, 125, or 150. In the embodiments of the present disclosure, the mass fraction of the first component may represent a sum of a mass fraction of the vinyl silicone rubber and a mass fraction of the cross-linking agent.

In some embodiments, the mass fraction of the second component may be in a range of 0-20 and greater than 0. In some embodiments, the mass fraction of the second component may be in a range of 2-18. In some embodiments, the mass fraction of the second component may be in a range of 5-15. In some embodiments, the mass fraction of the second component may be in a range of 8-12. In some embodiments, the mass fraction of the second component may be in a range of 10-20. In some embodiments, the mass fraction of the second component may be in a range of 10-15. In some embodiments, the mass fraction of the second component may be 5, 8, 10, 12, 14, or 15.

In some embodiments, a mass ratio of the first component to the second component may be (5-15):1. In some embodiments, the mass ratio of the first component to the second component may be (6-14):1. In some embodiments, the mass ratio of the first component to the second component may be (7-13):1. In some embodiments, the mass ratio of the first component to the second component may be (8-12):1. In some embodiments, the mass ratio of the first component to the second component may be (9-11):1. In some embodiments, the mass ratio of the first component to the second component may be 5:1, 8:1, 10:1, 12:1, or 15:1. For example, when the two-component addition type liquid silicone rubber is the two-component RTV liquid silicone rubber RTV630 or RTV615 produced by Momentive, the mass ratio of the first component to the second component may be 10:1. As another example, according to mass fraction, the first component may be 100 parts, and the second component may be 10 parts.

In some embodiments, when the two-component addition type liquid silicone rubber is the two-component RTV liquid silicone rubber RTV630 or RTV615 produced by Momentive, during use, the first component and the second component may be mixed well in the preset ratio (e.g., the mass ratio of the first component to the second component may be 10:1), then placed at the room temperature or high temperature (e.g., 30° C.-100° C., or 50° C.-70° C.) for a preset time (e.g., 12 h-60 h, or 36 h-60 h, or 48 h), and cured to obtain the RTV630 silicone rubber or the RTV615 silicone rubber. For example, the first component and the second component may be fully mixed in the preset ratio, then placed at the room temperature (e.g., 20° C.-30° C.) for 24 h-48 h, and cured to obtain the RTV630 silicone rubber or the RTV615 silicone rubber. As another example, the first component and the second component may be fully mixed in the preset ratio, then placed at the high temperature (e.g., greater than 30° C. and not greater than 100° C., or 50° C.-70° C.) for 12 h-24 h, and cured to obtain the RTV630 silicone rubber or the RTV615 silicone rubber.

In some embodiments, the raw material that can form the modified material may include a third component and a fourth component.

In some embodiments, the third component may include at least one of “a butadiene compound and an acrylonitrile compound”, a butadiene compound, or a fluorine-containing carbon chain. The “butadiene compound and the acrylonitrile compound” may represent that both the butadiene compound and the acrylonitrile compound are included.

In some embodiments, a structural formula of the butadiene compound may be shown in Formula II,

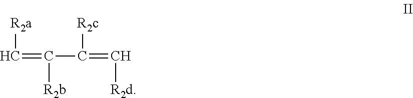

R₂a, R₂b, R₂c, R₂d may be independently selected from H, substituted or unsubstituted C₁-C₃ straight chain or branched chain alkyl, substituted or unsubstituted C₆-C₂₀ aryl. In some embodiments, in the structure shown in Formula II, one or more of R₂a, R₂b, R₂c, and R₂d may be selected from H. In some embodiments, the butadiene compound may be 1,3-butadiene.

In some embodiments, a structural formula of the acrylonitrile compound may be shown in formula III

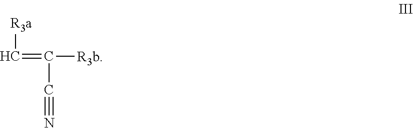

R₃a and R₃b may be independently selected from H, substituted or unsubstituted C₁-C₃ straight chain or branched chain alkyl, substituted or unsubstituted C₆-C₂₀ aryl. In some embodiments, in the structure shown in Formula III, R₃a and/or R₃b may be selected from H. In some embodiments, the acrylonitrile compound may be acrylonitrile.

In some embodiments, a structural formula of the fluorine-containing carbon chain may be shown in Formula IV,

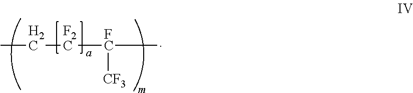

m may not be less than 200, a may be in a range of 1-8, and a may be an integer. In some embodiments, m may be in a range of 200-1000. For example, m may be 200, 300, 500, 800, or 1000. In some embodiments, a may be in a range of 1-5, and a may be an integer. For example, a may be 1, 2, 3, 4, or 5. In some embodiments, the structural formula of the fluorine-containing carbon chain may be

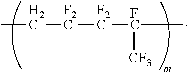

In some embodiments, when the third component includes the butadiene compound and the acrylonitrile compound, the fourth component may include a catalyst capable of catalyzing a reaction (e.g., an addition reaction) of the butadiene compound and the acrylonitrile compound. In some embodiments, when the third component includes the butadiene compound, the fourth component may include a catalyst capable of catalyzing a reaction (e.g., an addition reaction) of the butadiene compound. In some embodiments, when the third component includes a fluorine-containing carbon chain, the fourth component may include aziridine or polycarbodiimide.

In some embodiments, when the third component includes “the butadiene compound and the acrylonitrile compound” or the butadiene compound, the fourth component may include at least one of transition metals of Group VIII of the periodic table, a compound thereof, or a complex thereof. For example, the fourth component may include platinum, a platinum-containing compound, or a platinum-containing complex. In some embodiments, the fourth component may include organotin or organobismuth. For example, the organotin may be dibutyltin dilaurate or stannous octoate.

In some embodiments, the compound or the complex of the transition metals of Group VIII of the periodic table in the fourth component may be used as a catalyst or a curing agent instead of the filler. Accordingly, an amount of the compound or the complex of the transition metals of Group VIII of the periodic table in the fourth component may be a catalytic amount, while an amount of the added filler may be generally large, even exceed the amount of the silicone rubber (e.g., the RTV silicone rubber). In some embodiments, the compound or the complex of the transition metals of Group VIII of the periodic table in the fourth component may be a liquid, while filler may generally exist in a powder form.

In some embodiments, when the third component includes the fluorine-containing carbon chain, the fourth component may further include a catalyst. In some embodiments, the catalyst may include at least one of transition metals of Group VIII of the periodic table, a compound thereof, or a complex thereof. For example, the catalyst may include platinum, a platinum-containing compound, or a platinum-containing complex. In some embodiments, the catalyst may include organotin or organobismuth. In some embodiments, the organotin may be dibutyltin dilaurate or stannous octoate.

In some embodiments, the modified material may be at least one of nitrile rubber, cis-polybutadiene, or fluoro rubber. Accordingly, the raw material that can form the modified material may be at least one of a two-component raw material that can form the nitrile rubber, a two-component raw material that can form the cis-polybutadiene, or a two-component raw material that can form the fluoro rubber.

In some embodiments, in the two-component raw material that can form the nitrile rubber, the third component may include the butadiene compound and the acrylonitrile compound, and the fourth component may include a catalyst such as organotin or organobismuth. Merely by way of example, the organotin may be dibutyltin dilaurate or stannous octoate. In some embodiments, a mass percentage of acrylonitrile in the nitrile rubber may be in a range of 42%-46%. In some embodiments, the mass percentage of acrylonitrile in the nitrile rubber may be in a range of 36%-41%. In some embodiments, the mass percentage of acrylonitrile in the nitrile rubber may be in a range of 31%-35%. In some embodiments, the mass percentage of acrylonitrile in the nitrile rubber may be in a range of 25%-30%. In some embodiments, the mass percentage of acrylonitrile in the nitrile rubber may be in a range of 18%-24%. In some embodiments, the two-component raw material that can form the nitrile rubber may be packaged independently by two packaging devices (e.g., packaging barrels). During use, the two-component raw material that can form the nitrile rubber may be fully mixed in a preset ratio (e.g., determined according to the respective mass fractions of the two components), then placed at room temperature or high temperature (e.g., 30° C.-100° C., or 50° C.-70° C.) for a preset time (e.g., 12 h-60 h, or 36 h-60 h, or 48 h), and cured to obtain the nitrile rubber. For example, the two-component raw material that can form the nitrile rubber may be fully mixed in the preset ratio, and placed at the room temperature (e.g., 20° C.-30° C.) for 24 h-48 h, and cured to obtain the nitrile rubber. As another example, the two-component raw material that can form the nitrile rubber may be fully mixed in the preset ratio, then placed at the high temperature (e.g., greater than 30° C. and not greater than 100° C., or 50° C.-70° C.) for 12 h-24 h, and cured to obtain the nitrile rubber. In some embodiments, the two-component raw material that can form the nitrile rubber may be commercially available. For example, the two-component raw material that can form the nitrile rubber may be purchased from the product of the model LNBR820 produced by Shandong Wang Brothers Plastic Technology Co., Ltd.

In some embodiments, in the two-component raw material that can form the cis-polybutadiene, the third component may include the butadiene compound, and the fourth component may include a catalyst such as organotin or organobismuth. In some embodiments, the two-component raw material that can form the cis-polybutadiene may be packaged independently by two packaging devices (e.g., packaging barrels). During use, the two-component raw material that can form the cis-polybutadiene may be fully mixed in a preset ratio (e.g., determined according to the respective mass fractions of the two components), then placed at room temperature or high temperature (e.g., 30° C.-100° C., or 50° C.-70° C.) for a preset time (e.g., 12 h-60 h, or 36 h-60 h, or 48 h), and cured to obtain the cis-polybutadiene. For example, the two-component raw material that can form the cis-polybutadiene may be fully mixed in the preset ratio, then placed at the room temperature (e.g., 20° C.-30° C.) for 24 h-48 h, and then cured to obtain the cis-polybutadiene. As another example, the two-component raw material that can form the cis-polybutadiene may be fully mixed in the preset ratio, then placed at the high temperature (e.g., greater than 30° C. and not greater than 100° C., or 50° C.-70° C.) for 12 h-24 h, and cured to obtain the cis-polybutadiene. In some embodiments, the two-component raw material that can form the cis-polybutadiene may be commercially available. For example, the two-component raw material that can form the cis-polybutadiene may be purchased from the product of the model PBR-4040 produced by Yuyao Huihong Plastic Factory.

In some embodiments, in the two-component raw material that can form the fluoro rubber, the third component may include the fluorine-containing carbon chain, and the fourth component may include aziridine or polycarbodiimide. In some embodiments, the two-component raw material that can form the fluoro rubber may be packaged independently by two packaging devices (e.g., packaging barrels). During use, the two-component raw material that can form the fluoro rubber may be fully mixed in a preset ratio (e.g., determined according to the respective mass fractions of the two components), then placed at room temperature or high temperature (e.g., 30° C.-100° C., or 50° C.-70° C.) for a preset time (e.g., 12 h-60 h, 36 h-60 h, or 48 h), and cured to obtain the fluoro rubber. For example, the two-component raw material that can form the fluoro rubber may be fully mixed in the preset ratio, then placed at the room temperature (e.g., 20° C.-30° C.) for 24 h-48 h, and cured to obtain the fluoro rubber. As another example, the two-component raw material that can form the fluoro rubber may be fully mixed in the preset ratio, then placed at the high temperature (e.g., greater than 30° C. and not greater than 100° C., or 50° C.-70° C.) for 12 h-24 h, and cured to obtain the fluoro rubber. In some embodiments, the two-component raw material that can form the fluoro rubber may be commercially available. For example, the two-component raw material that can form the fluoro rubber may be purchased from the product of the model FAQ-008 produced by Shanghai Silicon Mountain Polymer Material Co., Ltd.

In some embodiments, a mass fraction of the third component may be in a range of 25-150. In some embodiments, the mass fraction of the third component may be in a range of 50-150. In some embodiments, the mass fraction of the third component may be in a range of 100-150. In some embodiments, the mass fraction of the third component may be in a range of 25-125. In some embodiments, the mass fraction of the third component may be in a range of 50-125. In some embodiments, the mass fraction of the third component may be in a range of 75-125. In some embodiments, the mass fraction of the third component may be in a range of 25-100. In some embodiments, the mass fraction of the third component may be in a range of 50-100. In some embodiments, the mass fraction of the third component may be in a range of 25-75. In some embodiments, the mass fraction of the third component may be 25, 50, 75, 100, 125, or 150.

In some embodiments, the mass fraction of the fourth component may be in a range of 0-20 and greater than 0. In some embodiments, the mass fraction of the fourth component may be in a range of 2-18. In some embodiments, the mass fraction of the fourth component may be in a range of 4-16. In some embodiments, the mass fraction of the fourth component may be in a range of 5-10. In some embodiments, the mass fraction of the fourth component may be 5, 8, 10, 12, 15, or 20.

In some embodiments, a mass ratio of the third component to the fourth component may be (5-15):1. In some embodiments, the mass ratio of the third component to the fourth component may be (6-14):1. In some embodiments, the mass ratio of the third component to the fourth component may be (7-13):1. In some embodiments, the mass ratio of the third component to the fourth component may be (8-12):1. In some embodiments, the mass ratio of the third component to the fourth component may be (9-11):1. In some embodiments, the mass ratio of the third component to the fourth component may be 5:1, 8:1, 10:1, 12:1, or 15:1. For example, according to mass fraction, the third component may be 100 parts, and the fourth component may be 10 parts. As another example, according to mass fraction, the third component may be 50 parts, and the fourth component may be 5 parts.

In the embodiments of the present disclosure, the modified silicone rubber may be prepared by fully mixing the raw material that can form the silicone rubber and the raw material that can form the modified material. The modified silicone rubber may include a large amount of Si—O bonds, methyl, and a small amount of vinyl. In some embodiments, the modified silicone rubber may further include at least one of an acrylonitrile group, butadienyl, or a fluorine atom.

In some embodiments, a mass ratio of the first component, the second component, the third component, and the fourth component may be 10:1:(5-10):(0.5-1). In some embodiments, the mass ratio of the first component, the second component, the third component, and the fourth component may be 10:1:(6-9):(0.5-1). In some embodiments, the mass ratio of the first component, the second component, the third component, and the fourth component may be 10:1:(7-8):(0.5-1). In some embodiments, the mass ratio of the first component, the second component, the third component, and the fourth component may be 10:1:(5-10):(0.6-0.9). In some embodiments, the mass ratio of the first component, the second component, the third component, and the fourth component may be 10:1:(5-10):(0.7-0.8). In some embodiments, the mass ratio of the first component, the second component, the third component, and the fourth component may be 10:1:10:1, 10:1:10:0.5, 10:1:5:0.5, or 10:1:5:1.

In some embodiments, a mass ratio of the first component to the third component may be (1-2):1. In some embodiments, the mass ratio of the first component to the third component may be (1.2-1.8):1. In some embodiments, the mass ratio of the first component to the third component may be (1.4-1.6):1. In some embodiments, the mass ratio of the first component to the third component may be 1:1, 1.2:1, 1.4:1, 1.6:1, 1.8:1, or 2:1.

In some embodiments, the raw material that can form the silicone rubber may be two-component RTV liquid silicone rubber RTV615, according to mass fraction, the third component may be 100 parts, and the fourth component may be 10 parts. In some embodiments, the raw material that can form the silicone rubber may be two-component RTV liquid silicone rubber RTV630, according to mass fraction, the third component may be 50 parts, and the fourth component may be 5 parts.

In some embodiments, according to mass fraction, the composition of raw materials for preparing the modified silicone rubber may include 50-150 parts of the first component, 0-20 parts of the second component, 25-150 parts of the third component, and 0-20 parts of the fourth component, wherein the mass fractions of the second component and the fourth component may not be zero. In some embodiments, according to mass fraction, the composition of raw materials for preparing the modified silicone rubber may include 100 parts of the first component, 10 parts of the second component, 50-100 parts of the third component, and 0-10 parts of the fourth component, wherein the mass fraction of the fourth component may not be zero. In some embodiments, according to mass fraction, the composition of raw materials for preparing the modified silicone rubber may include 100 parts of the first component of the RTV silicone rubber, 10 parts of the second component of the RTV silicone rubber, 50-100 parts of the third component, and 0-10 parts of the fourth component, wherein the mass fraction of the fourth component may not be zero. In some embodiments, according to mass fraction, the composition of raw materials for preparing the modified silicone rubber may include 100 parts of the first component of the RTV silicone rubber, 10 parts of the second component of the RTV silicone rubber, 50-100 parts of “butadiene and acrylonitrile,” and 0-10 parts of “organotin or organobismuth,” wherein the mass fraction of the “organotin or organobismuth” may not be zero. In some embodiments, according to mass fraction, the composition of raw materials for preparing the modified silicone rubber may include 100 parts of the first component of the RTV silicone rubber, 10 parts of the second component of the RTV silicone rubber, 50-100 parts of butadiene, and 0-10 parts of “organotin or organobismuth,” wherein the mass fraction of “organotin or organobismuth” may not be zero. In some embodiments, according to mass fraction, the composition of raw materials for preparing the modified silicone rubber may include 100 parts of first component of the RTV silicone rubber, 10 parts of second component of the RTV silicone rubber, 50-100 parts of fluorine-containing carbon chains, and 0-10 parts of “aziridine or polycarbodiimide,” wherein the mass fraction of “aziridine or polycarbodiimide” may not be zero.

In some embodiments, according to mass fraction, the composition of raw materials for preparing the modified silicone rubber and an amount thereof may be any one of No 1-8 in Table 1.

TABLE 1
Composition of raw materials for preparing the
modified silicone rubber and amount thereof

| | Type of | | | Type of | | |
|---|---|---|---|---|---|---|
| | silicone | | | modified | | |
| | rubber | Amount of | Amount of | material | Amount of | Amount of |
| | that can | the first | the second | that can | the third | the fourth |
| No. | be formed | component | component | be formed | component | component |
| 1 | RTV630 | 100 | 10 | Fluoro rubber | 50 | 5 |
| 2 | RTV630 | 100 | 10 | Cis- | 50 | 5 |
| | | | | polybutadiene | | |
| 3 | RTV630 | 100 | 10 | Nitrile rubber | 50 | 5 |
| 4 | RTV630 | 100 | 10 | Nitrile rubber | 100 | 10 |
| 5 | RTV615 | 100 | 10 | Fluoro rubber | 50 | 5 |
| 6 | RTV615 | 100 | 10 | Cis- | 50 | 5 |
| | | | | polybutadiene | | |
| 7 | RTV615 | 100 | 10 | Nitrile rubber | 50 | 5 |
| 8 | RTV615 | 100 | 10 | Nitrile rubber | 100 | 10 |

It should be noted that the above descriptions are for illustration and description purposes only, and does not limit the scope of the application of the present disclosure. Various modifications and variations may be made by those skilled in the art under the guidance of the present disclosure. However, such modifications and variations remain within the scope of the present disclosure.

The embodiments of the present disclosure further provide a preparation method of modified silicone rubber. The preparation method may include mixing a composition of raw materials for preparing the modified silicone rubber and obtaining the modified silicone rubber by curing and molding the mixture. More description regarding the composition of raw materials for preparing the modified silicone rubber may be found elsewhere in the present disclosure, which is not repeated herein.

In some embodiments, a curing temperature of the curing may be determined according to properties of the raw materials in the composition of raw materials for preparing the modified silicone rubber. For example, when the raw materials in the composition of raw materials for preparing the modified silicone rubber can be cured at room temperature, the curing temperature may be the room temperature. As another example, the curing temperature may be in a range of 30° C.-100° C. As yet another example, the curing temperature may be in a range of 50° C.-70° C. In the embodiments of the present disclosure, the room temperature may be 25° C.±5° C., or in a range of 20° C.-30° C.

In some embodiments, a curing time of the curing may be determined according to the properties of the raw materials in the composition of raw materials for preparing the modified silicone rubber, and a complete reaction should be ensured. For example, the curing time may be in a range of 12 h-60 h. As another example, the curing time may be in a range of 36 h-60 h. As yet another example, the curing time may be 48 h. For example, the raw materials in the composition of raw materials for preparing the modified silicone rubber may be fully mixed in a preset ratio, then placed at the room temperature (e.g., 20° C.-30° C.) for 24 h-48 h, and cured and molded to obtain the modified silicone rubber. As another example, the raw materials in the composition of raw materials for preparing the modified silicone rubber may be fully mixed in the preset ratio, then placed at a high temperature (e.g., greater than 30° C. and not greater than 100° C., or in a range of 50° C.-70° C.) for 12 h-24 h, and cured and molded to obtain the modified silicone rubber.

In some embodiments, when the composition of raw materials for preparing the modified silicone rubber is in a liquid state, the curing may be performed in a mold.

In some embodiments, the preparation method may further include performing defoaming after mixing the composition of raw materials for preparing the modified silicone rubber and before performing the curing.

In some embodiments, the preparation method of the modified silicone rubber may include: (1) obtaining a mixture A by mixing a first component and a third component; (2) obtaining a mixture B by mixing the mixture A, a second component and a fourth component; and (3) obtaining the modified silicone rubber by curing and molding the mixture B. For example, the preparation method of the modified silicone rubber may include: according to mass fraction, weighing 100 parts of the first component of the RTV silicone rubber and 0-100 (not zero) parts of the third component and mixing well (e.g., in a beaker), then adding 10 parts of the second component of the RTV silicone rubber and 0-10 (not zero) parts of the fourth component and mixing well to obtain a mixed solution, pouring the mixed solution into a mold, and curing and molding at the room temperature or a high temperature (e.g., 30° C.-100° C., or 50° C.-70° C.) to obtain the modified silicone rubber.

The embodiments of the present disclosure further provide modified silicone rubber prepared by the preparation method of the modified silicone rubber.

In some embodiments, a viscosity of the modified silicone rubber may be greater than or equal to 10000 mpa·s. In some embodiments, when the raw material that can form the silicone rubber is the raw material that can form the RTV615 silicone rubber, the viscosity of the modified silicone rubber may be in a range of 10000 mpa·s-30000 mpa·s. In some embodiments, when the raw material that can form the silicone rubber is the raw material that can form the RTV630 silicone rubber, the viscosity of the modified silicone rubber may be greater than or equal to 150000 mpa·s.

The embodiments of the present disclosure further provide modified silicone rubber which can be a random copolymer composed of structural units that are in any of the following conditions: condition 1, structural units shown in a general formula I-1 and a general formula II-1; condition 2, structural units shown in the general formula I-1, the general formula II-1, and a general formula III-1; and condition3, structural units shown in the general formula I-1, a general formula IV-1, and “a general formula V or a general formula VI”, i.e., the structural units shown in the general formula I-1, the general formula IV-1, and the general formula V, or the structural units shown in the general formula I-1, the general formula IV-1, and the general formula VI.

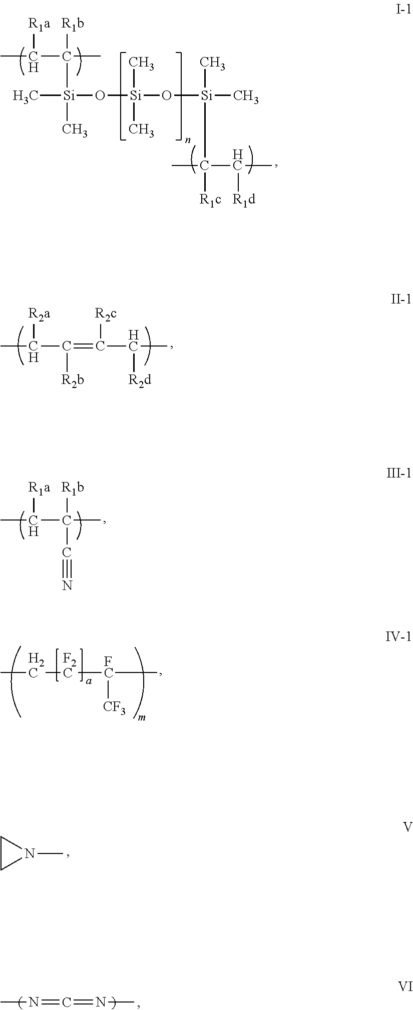

wherein definitions of R₁a, R₁b, R₁c, R₁d, R₂a, R₂b, R₂c, R₂d, R₃a, R₃b, n, m, and a may be as described above.

In some embodiments, an acoustic impedance of the modified silicone rubber in the embodiments of the present disclosure is greater than that of the silicone rubber obtained by the polymerization of the structural unit shown in the general formula I-1. A difference between the acoustic impedance of the modified silicone rubber in the embodiments of the present disclosure and the acoustic impedance of the silicone rubber obtained by the polymerization of the structural unit shown in the general formula I-1 may be greater than or equal to 0.05 Mrayl. In some embodiments, the difference between the acoustic impedance of the modified silicone rubber in the embodiments of the present disclosure and the acoustic impedance of the silicone rubber obtained by the polymerization of the structural unit shown in the general formula I-1 may be greater than or equal to 0.05 Mrayl and less than or equal to 0.5 Mrayl. In some embodiments, the difference between the acoustic impedance of the modified silicone rubber in the embodiments of the present disclosure and the acoustic impedance of the silicone rubber obtained by the polymerization of the structural unit shown in the general formula I-1 may be greater than or equal to 0.1 Mrayl and less than or equal to 0.45 Mrayl. In some embodiments, the difference between the acoustic impedance of the modified silicone rubber in the embodiments of the present disclosure and the acoustic impedance of the silicone rubber obtained by the polymerization of the structural unit shown in the general formula I-1 may be greater than or equal to 0.15 Mrayl and less than or equal to 0.4 Mrayl. In some embodiments, the difference between the acoustic impedance of the modified silicone rubber in the embodiments of the present disclosure and the acoustic impedance of the silicone rubber obtained by the polymerization of the structural unit shown in the general formula I-1 may be greater than or equal to 0.2 Mrayl and less than or equal to 0.35 Mrayl. In some embodiments, the difference between the acoustic impedance of the modified silicone rubber in the embodiments of the present disclosure and the acoustic impedance of the silicone rubber obtained by the polymerization of the structural unit shown in the general formula I-1 may be greater than or equal to 0.25 Mrayl and less than or equal to 0.3 Mrayl. For example, the difference between the acoustic impedance of the modified silicone rubber in the embodiments of the present disclosure and the acoustic impedance of the silicone rubber obtained by the polymerization of the structural unit shown in the general formula I-1 may be 0.06 Mrayl, 0.09 Mrayl, 0.12 Mrayl, 0.20 Mrayl, 0.21 Mrayl, 0.26 Mrayl, 0.31 Mrayl, 0.32 Mrayl, 0.35 Mrayl, 0.4 Mrayl, 0.45 Mrayl, or 0.5 Mrayl.

In the embodiments of the present disclosure, since the acoustic impedances of the silicone rubber obtained by different degrees of polymerization of the structural unit shown in the general formula I-1 may be different, the comparison of the acoustic impedance of the modified silicone rubber in the embodiments of the present disclosure and the acoustic impedance of the silicone rubber obtained by the polymerization of the structural unit shown in the general formula I-1 may be performed under a condition that the modified silicone rubber in the embodiments of the present disclosure and the silicone rubber obtained by the polymerization of the structural unit shown in the general formula I-1 have the same silicone rubber structural units.

In some embodiments, when the silicone rubber component of the modified silicone rubber in the embodiments of the present disclosure is the RTV630 silicone rubber, a difference between the acoustic impedance of the modified silicone rubber in the embodiments of the present disclosure and the acoustic impedance of the RTV630 silicone rubber (e.g., the silicone rubber obtained by the polymerization of the structural unit shown in the general formula I-1) may be greater than or equal to 0.06 Mrayl and less than or equal to 0.3 Mrayl. In some embodiments, when the silicone rubber component of the modified silicone rubber in the embodiments of the present disclosure is the RTV630 silicone rubber, the difference between the acoustic impedance of the modified silicone rubber in the embodiments of the present disclosure and the acoustic impedance of the RTV630 silicone rubber may be greater than or equal to 0.1 Mrayl and less than or equal to 0.25 Mrayl. In some embodiments, when the silicone rubber component of the modified silicone rubber in the embodiments of the present disclosure is the RTV630 silicone rubber, the difference between the acoustic impedance of the modified silicone rubber in the embodiments of the present disclosure and the acoustic impedance of the RTV630 silicone rubber may be greater than or equal to 0.15 Mrayl and less than or equal to 0.2 Mrayl. For example, when the silicone rubber component of the modified silicone rubber in the embodiments of the present disclosure is the RTV630 silicone rubber, the difference between the acoustic impedance of the modified silicone rubber in the embodiments of the present disclosure and the acoustic impedance of the RTV630 silicone rubber may be 0.06 Mrayl, 0.09 Mrayl, 0.12 Mrayl, or 0.20 Mrayl.

In some embodiments, when the silicone rubber component of the modified silicone rubber in the embodiments of the present disclosure is the RTV615 silicone rubber, a difference between the acoustic impedance of the modified silicone rubber in the embodiments of the present disclosure and the acoustic impedance of the RTV615 silicone rubber (e.g., the silicone rubber obtained by the polymerization of the structural unit shown in the general formula I-1) may be greater than or equal to 0.2 Mrayl and less than or equal to 0.5 Mrayl. In some embodiments, when the silicone rubber component of the modified silicone rubber in the embodiments of the present disclosure is the RTV615 silicone rubber, the difference between the acoustic impedance of the modified silicone rubber in the embodiments of the present disclosure and the acoustic impedance of the RTV615 silicone rubber may be greater than or equal to 0.25 Mrayl and less than or equal to 0.45 Mrayl. In some embodiments, when the silicone rubber component of the modified silicone rubber in the embodiments of the present disclosure is the RTV615 silicone rubber, the difference between the acoustic impedance of the modified silicone rubber in the embodiments of the present disclosure and the acoustic impedance of the RTV615 silicone rubber may be greater than or equal to 0.3 Mrayl less than or equal to 0.4 Mrayl. In some embodiments, when the silicone rubber component of the modified silicone rubber in the embodiments of the present disclosure is the RTV615 silicone rubber, the difference between the acoustic impedance of the modified silicone rubber in the embodiments of the present disclosure and the acoustic impedance of the RTV615 silicone rubber may be greater than or equal to 0.32 Mrayl and less than or equal to 0.35 Mrayl. For example, when the silicone rubber component of the modified silicone rubber in the embodiments of the present disclosure is the RTV615 silicone rubber, the difference between the acoustic impedance of the modified silicone rubber in the embodiments of the present disclosure and the acoustic impedance of the RTV615 silicone rubber may be 0.21 Mrayl, 0.26 Mrayl, 0.31 Mrayl, or 0.32 Mrayl.

In some embodiments, the acoustic impedance of the modified silicone rubber in the embodiments of the present disclosure may be in a range of 1.25 MRayl-1.50 MRayl. In some embodiments, the acoustic impedance of the modified silicone rubber in the embodiments of the present disclosure may be in a range of 1.30 MRayl-1.50 MRayl. In some embodiments, the acoustic impedance of the modified silicone rubber in the embodiments of the present disclosure may be in a range of 1.35 MRayl-1.50 MRayl. In some embodiments, the acoustic impedance of the modified silicone rubber in the embodiments of the present disclosure may be in a range of 1.40 MRayl-1.50 MRayl. In some embodiments, the acoustic impedance of the modified silicone rubber in the embodiments of the present disclosure may be in a range of 1.45 MRayl-1.50 MRayl. For example, the acoustic impedance of the modified silicone rubber in the embodiments of the present disclosure may be 1.26 MRayl, 1.31 MRayl, 1.36 MRayl, 1.37 MRayl, 1.39 MRayl, 1.42 MRayl, or 1.50 MRayl.

In some embodiments, when the silicone rubber component of the modified silicone rubber in the embodiments of the present disclosure is the RTV630 silicone rubber, the acoustic impedance of the modified silicone rubber may be in a range of 1.30 MRayl-1.50 MRayl. In some embodiments, when the silicone rubber component of the modified silicone rubber in the embodiments of the present disclosure is the RTV630 silicone rubber, the acoustic impedance of the modified silicone rubber may be in a range of 1.35 MRayl-1.50 MRayl. In some embodiments, when the silicone rubber component of the modified silicone rubber in the embodiments of the present disclosure is the RTV630 silicone rubber, the acoustic impedance of the modified silicone rubber may be in a range of 1.40 MRayl-1.50 MRayl. In some embodiments, when the silicone rubber component of the modified silicone rubber in the embodiments of the present disclosure is the RTV630 silicone rubber, the acoustic impedance of the modified silicone rubber may be in a range of 1.45 MRayl-1.50 MRayl. For example, when the silicone rubber component of the modified silicone rubber in the embodiments of the present disclosure is the RTV630 silicone rubber, the acoustic impedance of the modified silicone rubber may be 1.36 MRayl, 1.39 MRayl, 1.42 MRayl, or 1.50 MRayl.

In some embodiments, when the silicone rubber component of the modified silicone rubber in the embodiments of the present disclosure is the RTV615 silicone rubber, the acoustic impedance of the modified silicone rubber may be in a range of 1.20 MRayl-1.40 MRayl. In some embodiments, when the silicone rubber component of the modified silicone rubber in the embodiments of the present disclosure is the RTV615 silicone rubber, the acoustic impedance of the modified silicone rubber may be in a range of 1.25 MRayl-1.40 MRayl. In some embodiments, when the silicone rubber component of the modified silicone rubber in the embodiments of the present disclosure is the RTV615 silicone rubber, the acoustic impedance of the modified silicone rubber may be in a range of 1.30 MRayl-1.40 MRayl. In some embodiments, when the silicone rubber component of the modified silicone rubber in the embodiments of the present disclosure is the RTV615 silicone rubber, the acoustic impedance of the modified silicone rubber may be in a range of 1.35 MRayl-1.40 MRayl. For example, when the silicone rubber component of the modified silicone rubber in the embodiments of the present disclosure is the RTV615 silicone rubber, the acoustic impedance of the modified silicone rubber may be 1.26 MRayl, 1.31 MRayl, 1.36 MRayl, 1.37 MRayl, or 1.40 MRayl.

In the embodiments of the present disclosure, the acoustic impedance of the modified silicone rubber may be obtained by an oscilloscope through hydroacoustic measurement. For example, a density of a modified silicone rubber sample may be calculated through a density formula: ρ=m/V, wherein m is the mass of the modified silicone rubber sample, and V is the volume of the modified silicone rubber sample. Further, a material sound velocity of the modified silicone rubber sample may be obtained by a water insertion method, and the acoustic impedance of the modified silicone rubber sample may be calculated according to a calculation formula as follows:

C=(I₁−I₂)C₀/(ΔtC₀+(I₁−I₂));

Z=ρC;

where C represents the material sound velocity of modified silicone rubber, I1 is a thickness of modified silicone rubber sample 1, I₂ is a thickness of modified silicone rubber sample 2, Δt is a time difference of sound propagation caused by the insertion of the modified silicone rubber sample 1 and the modified silicone rubber sample 2, C₀ is a sound velocity in water, and Z is the acoustic impedance of the modified silicone rubber sample.

In some embodiments, a sound attenuation of the modified silicone rubber in the embodiments of the present disclosure may be greater than or less than a sound attenuation of the silicone rubber obtained by the polymerization of the structural unit shown in the general formula I-1. In the embodiments of the present disclosure, an absolute value of a difference between the sound attenuation of the modified silicone rubber and the sound attenuation of the silicone rubber obtained by the polymerization of the structural unit shown in the general formula I-1 may be in a range of 0.0-15.0 dB/cm. In some embodiments, the absolute value of the difference between the sound attenuation of the modified silicone rubber in the embodiments of the present disclosure and the sound attenuation of the silicone rubber obtained by the polymerization of the structural unit shown in the general formula I-1 may be in a range of 1.0 dB/cm-14.0 dB/cm. In some embodiments, the absolute value of the difference between the sound attenuation of the modified silicone rubber in the embodiments of the present disclosure and the sound attenuation of the silicone rubber obtained by the polymerization of the structural unit shown in the general formula I-1 may be in a range of 2.0 dB/cm-13.0 dB/cm. In some embodiments, the absolute value of the difference between the sound attenuation of the modified silicone rubber in the embodiments of the present disclosure and the sound attenuation of the silicone rubber obtained by the polymerization of the structural unit shown in the general formula I-1 may be in a range of 3.0 dB/cm-12.0 dB/cm. In some embodiments, the absolute value of the difference between the sound attenuation of the modified silicone rubber in the embodiments of the present disclosure and the sound attenuation of the silicone rubber obtained by the polymerization of the structural unit shown in the general formula I-1 may be in a range of 4.0 dB/cm-11.0 dB/cm. In some embodiments, the absolute value of the difference between the sound attenuation of the modified silicone rubber in the embodiments of the present disclosure and the sound attenuation of the silicone rubber obtained by the polymerization of the structural unit shown in the general formula I-1 may be in a range of 5.0 dB/cm-10.0 dB/cm. In some embodiments, the absolute value of the difference between the sound attenuation of the modified silicone rubber in the embodiments of the present disclosure and the sound attenuation of the silicone rubber obtained by the polymerization of the structural unit shown in the general formula I-1 may be in a range of 6.0 dB/cm-9.0 dB/cm. In some embodiments, the absolute value of the difference between the sound attenuation of the modified silicone rubber in the embodiments of the present disclosure and the sound attenuation of the silicone rubber obtained by the polymerization of the structural unit shown in the general formula I-1 may be in a range of 7.0 dB/cm-8.0 dB/cm. For example, the absolute value of the difference between the sound attenuation of the modified silicone rubber in the embodiments of the present disclosure and the sound attenuation of the silicone rubber obtained by the polymerization of the structural unit shown in the general formula I-1 may be 10.0 dB/cm, 8.0 dB/cm, 2.9 dB/cm, 2.3 dB/cm, 1.3 dB/cm, 9.6 dB/cm, 11.0 dB/cm, or 13.7 dB/cm.

In the embodiments of the present disclosure, since the sound attenuation of the silicone rubber obtained by different degrees of polymerization of the structural unit shown in the general formula I-1 may be different, the comparison of the sound attenuation of the modified silicone rubber in the embodiments of the present disclosure and the sound attenuation of the silicone rubber obtained by the polymerization of the structural unit shown in the general formula I-1 may be performed under a condition that the modified silicone rubber in the embodiments of the present disclosure and the silicone rubber obtained by the polymerization of the structural unit shown in the general formula I-1 have the same silicone rubber structural units.

In some embodiments, when the silicone rubber component of the modified silicone rubber in the embodiments of the present disclosure is the RTV630 silicone rubber, an absolute value of a difference between the sound attenuation of the modified silicone rubber and the sound attenuation of the RTV630 silicone rubber (e.g., the silicone rubber obtained by the polymerization of the structural unit shown in the general formula I-1) may be in a range of 0.0-15.0 dB/cm. In some embodiments, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be in a range of 2.0 dB/cm-15.0 dB/cm. In some embodiments, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be in a range of 4.0 dB/cm-15.0 dB/cm. In some embodiments, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be in a range of 6.0 dB/cm-15.0 dB/cm. In some embodiments, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be in a range of 8.0 dB/cm-15.0 dB/cm. In some embodiments, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be in a range of 10.0 dB/cm-15.0 dB/cm. In some embodiments, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be in a range of 12.0 dB/cm-15.0 dB/cm. In some embodiments, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be in a range of 14.0 dB/cm-15.0 dB/cm. For example, when the silicone rubber component of the modified silicone rubber in the embodiments of the present disclosure is the RTV630 silicone rubber, the absolute value of the difference between the sound attenuation of the modified silicone rubber and the sound attenuation of the RTV630 silicone rubber may be 10.0 dB/cm, 8.0 dB/cm, 1.3 dB/cm, or 11.0 dB/cm.

In some embodiments, when the silicone rubber component of the modified silicone rubber in the embodiments of the present disclosure is the RTV615 silicone rubber, an absolute value of a difference between the sound attenuation of the modified silicone rubber and the sound attenuation of the RTV615 silicone rubber (i.e., the silicone rubber obtained by the polymerization of the structural unit shown in the general formula I-1) may be in a range of 0.0-15.0 dB/cm. In some embodiments, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be in a range of 2.0 dB/cm-15.0 dB/cm. In some embodiments, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be in a range of 4.0 dB/cm-15.0 dB/cm. In some embodiments, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be in a range of 6.0 dB/cm-15.0 dB/cm. In some embodiments, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be in a range of 8.0 dB/cm-15.0 dB/cm. In some embodiments, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be in a range of 10.0 dB/cm-15.0 dB/cm. In some embodiments, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be in a range of 12.0 dB/cm-15.0 dB/cm. In some embodiments, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be in a range of 14.0 dB/cm-15.0 dB/cm. For example, when the silicone rubber component of the modified silicone rubber in the embodiments of the present disclosure is the RTV615 silicone rubber, the absolute value of the difference between the sound attenuation of the modified silicone rubber and the sound attenuation of the RTV615 silicone rubber may be 2.9 dB/cm, 2.3 dB/cm, 9.6 dB/cm, or 13.7 dB/cm.

In the embodiments of the present disclosure, a sound attenuation coefficient (which may be referred to as a sound attenuation for short) of the modified silicone rubber at a frequency of 5 MHz may not be greater than 42.0 dB/cm. In some embodiments, the sound attenuation of the modified silicone rubber in the embodiments of the present disclosure at the frequency of 5 MHz may not be greater than 38.0 dB/cm. In some embodiments, the sound attenuation of the modified silicone rubber in the embodiments of the present disclosure at the frequency of 5 MHz may not be greater than 34.0 dB/cm. In some embodiments, the sound attenuation of the modified silicone rubber in the embodiments of the present disclosure at the frequency of 5 MHz may not be greater than 30.0 dB/cm. In some embodiments, the sound attenuation of the modified silicone rubber in the embodiments of the present disclosure at the frequency of 5 MHz may not be greater than 26.0 dB/cm. In some embodiments, the sound attenuation of the modified silicone rubber in the embodiments of the present disclosure at the frequency of 5 MHz may not be greater than 22.0 dB/cm. In some embodiments, the sound attenuation of the modified silicone rubber in the embodiments of the present disclosure at the frequency of 5 MHz may not be greater than 18.0 dB/cm. In some embodiments, the sound attenuation of the modified silicone rubber in the embodiments of the present disclosure at the frequency of 5 MHz may not be greater than 14.0 dB/cm. In some embodiments, the sound attenuation of the modified silicone rubber in the embodiments of the present disclosure at the frequency of 5 MHz may not be greater than 10.0 dB/cm. In some embodiments, the sound attenuation of the modified silicone rubber in the embodiments of the present disclosure at the frequency of 5 MHz may not be greater than 6.0 dB/cm. In some embodiments, the sound attenuation of the modified silicone rubber in the embodiments of the present disclosure at the frequency of 5 MHz may not be greater than 2.0 dB/cm.

In some embodiments, the sound attenuation of the modified silicone rubber in the embodiments of the present disclosure at the frequency of 5 MHz may be in a range of 12.0 dB/cm-42.0 dB/cm. In some embodiments, the sound attenuation of the modified silicone rubber in the embodiments of the present disclosure at the frequency of 5 MHz may be in a range of 12.0 dB/cm-38.0 dB/cm. In some embodiments, the sound attenuation of the modified silicone rubber in the embodiments of the present disclosure at the frequency of 5 MHz may be in a range of 12.0 dB/cm-34.0 dB/cm. In some embodiments, the sound attenuation of the modified silicone rubber in the embodiments of the present disclosure at the frequency of 5 MHz may be in a range of 12.0 dB/cm-30.0 dB/cm. In some embodiments, the sound attenuation of the modified silicone rubber in the embodiments of the present disclosure at the frequency of 5 MHz may be in a range of 12.0 dB/cm-26.0 dB/cm. In some embodiments, the sound attenuation of the modified silicone rubber in the embodiments of the present disclosure at the frequency of 5 MHz may be in a range of 12.0 dB/cm-22.0 dB/cm. In some embodiments, the sound attenuation of the modified silicone rubber in the embodiments of the present disclosure at the frequency of 5 MHz may be in a range of 12.0 dB/cm-18.0 dB/cm. In some embodiments, the sound attenuation of the modified silicone rubber in the embodiments of the present disclosure at the frequency of 5 MHz may be in a range of 12.0 dB/cm-14.0 dB/cm. For example, the sound attenuation of the modified silicone rubber in the embodiments of the present disclosure at the frequency of 5 MHz may be 12.5 dB/cm, 13.1 dB/cm, 20.0 dB/cm, 22.0 dB/cm, 25.0 dB/cm, 29.1 dB/cm, 31.3 dB/cm, or 41.0 dB/cm.

In some embodiments, when the silicone rubber component of the modified silicone rubber in the embodiments of the present disclosure is the RTV630 silicone rubber, the sound attenuation of the modified silicone rubber may be in a range of 20.0 dB/cm-42.0 dB/cm. In some embodiments, when the silicone rubber component of the modified silicone rubber in the embodiments of the present disclosure is the RTV630 silicone rubber, the sound attenuation of the modified silicone rubber may be in a range of 20.0 dB/cm-38.0 dB/cm. In some embodiments, when the silicone rubber component of the modified silicone rubber in the embodiments of the present disclosure is the RTV630 silicone rubber, the sound attenuation of the modified silicone rubber may be in a range of 20.0 dB/cm-34.0 dB/cm. In some embodiments, when the silicone rubber component of the modified silicone rubber in the embodiments of the present disclosure is the RTV630 silicone rubber, the sound attenuation of the modified silicone rubber may be in a range of 20.0 dB/cm-30.0 dB/cm. In some embodiments, when the silicone rubber component of the modified silicone rubber in the embodiments of the present disclosure is the RTV630 silicone rubber, the sound attenuation of the modified silicone rubber may be in a range of 20.0 dB/cm-26.0 dB/cm. In some embodiments, when the silicone rubber component of the modified silicone rubber in the embodiments of the present disclosure is the RTV630 silicone rubber, the sound attenuation of the modified silicone rubber may be in a range of 20.0 dB/cm-22.0 dB/cm. For example, when the silicone rubber component of the modified silicone rubber in the embodiments of the present disclosure is the RTV630 silicone rubber, the sound attenuation of the modified silicone rubber may be 20.0 dB/cm, 22.0 dB/cm, 31.3 dB/cm, or 41.0 dB/cm.

In some embodiments, when the silicone rubber component of the modified silicone rubber in the embodiments of the present disclosure is the RTV615 silicone rubber, the sound attenuation of the modified silicone rubber may be in a range of 12.0 dB/cm-42.0 dB/cm. In some embodiments, when the silicone rubber component of the modified silicone rubber in the embodiments of the present disclosure is the RTV615 silicone rubber, the sound attenuation of the modified silicone rubber may be in a range of 12.0 dB/cm-38.0 dB/cm. In some embodiments, when the silicone rubber component of the modified silicone rubber in the embodiments of the present disclosure is the RTV615 silicone rubber, the sound attenuation of the modified silicone rubber may be in a range of 12.0 dB/cm-34.0 dB/cm. In some embodiments, when the silicone rubber component of the modified silicone rubber in the embodiments of the present disclosure is the RTV615 silicone rubber, the sound attenuation of the modified silicone rubber may be in a range of 12.0 dB/cm-30.0 dB/cm. In some embodiments, when the silicone rubber component of the modified silicone rubber in the embodiments of the present disclosure is the RTV615 silicone rubber, the sound attenuation of the modified silicone rubber may be in a range of 12.0 dB/cm-26.0 dB/cm. In some embodiments, when the silicone rubber component of the modified silicone rubber in the embodiments of the present disclosure is the RTV615 silicone rubber, the sound attenuation of the modified silicone rubber may be in a range of 12.0 dB/cm-22.0 dB/cm. In some embodiments, when the silicone rubber component of the modified silicone rubber in the embodiments of the present disclosure is the RTV615 silicone rubber, the sound attenuation of the modified silicone rubber may be in a range of 12.0 dB/cm-18.0 dB/cm. In some embodiments, when the silicone rubber component of the modified silicone rubber in the embodiments of the present disclosure is the RTV615 silicone rubber, the sound attenuation of the modified silicone rubber may be in a range of 12.0 dB/cm-14.0 dB/cm. In some embodiments, when the silicone rubber component of the modified silicone rubber in the embodiments of the present disclosure is the RTV615 silicone rubber, the sound attenuation of the modified silicone rubber may be in a range of 20.0 dB/cm-42.0 dB/cm. For example, when the silicone rubber component of the modified silicone rubber in the embodiments of the present disclosure is the RTV615 silicone rubber, the sound attenuation of the modified silicone rubber may be 12.5 dB/cm, 13.1 dB/cm, 25.0 dB/cm, or 29.1 dB /cm.

In the embodiments of the present disclosure, the sound attenuation of the modified silicone rubber may be obtained by an oscilloscope through hydroacoustic measurement. For example, the sound attenuation coefficient of modified silicone rubber sample may be obtained by a water insertion method according to a calculation formula as follows:

α=(20 Ig(A₁/A₂))/(I₁−I₂)+α₀;

where I₁ is a thickness of modified silicone rubber sample 1, I₂ is a thickness of modified silicone rubber sample 2, α₀ is a sound attenuation coefficient in water, A₁ and A₂ are an amplitude of a pulse signal respectively received by the modified silicone rubber sample 1 and the modified silicone rubber sample 2, α is the sound attenuation coefficient of the modified silicone rubber sample in water.

In some embodiments, a hardness of the modified silicone rubber in the embodiments of the present disclosure may be greater than a hardness of the silicone rubber obtained by the polymerization of the structural unit shown in the general formula I-1. In the embodiments of the present disclosure, a difference between the hardness of the modified silicone rubber and the hardness of the silicone rubber obtained by the polymerization of the structural unit shown in the general formula I-1 may be in a range of 0-30 HA. In some embodiments, the difference between the hardness of the modified silicone rubber in the embodiments of the present disclosure and the hardness of the silicone rubber obtained by the polymerization of the structural unit shown in the general formula I-1 may be in a range of 2 HA-28 HA. In some embodiments, the difference between the hardness of the modified silicone rubber in the embodiments of the present disclosure and the hardness of the silicone rubber obtained by the polymerization of the structural unit shown in the general formula I-1 may be in a range of 4 HA-26 HA. In some embodiments, the difference between the hardness of the modified silicone rubber in the embodiments of the present disclosure and the hardness of the silicone rubber obtained by the polymerization of the structural unit shown in the general formula I-1 may be in a range of 6 HA-24 HA. In some embodiments, the difference between the hardness of the modified silicone rubber in the embodiments of the present disclosure and the hardness of the silicone rubber obtained by the polymerization of the structural unit shown in the general formula I-1 may be in a range of 8 HA-22 HA. In some embodiments, the difference between the hardness of the modified silicone rubber in the embodiments of the present disclosure and the hardness of the silicone rubber obtained by the polymerization of the structural unit shown in the general formula I-1 may be in a range of 10 HA-20 HA. In some embodiments, the difference between the hardness of the modified silicone rubber in the embodiments of the present disclosure and the hardness of the silicone rubber obtained by the polymerization of the structural unit shown in the general formula I-1 may be in a range of 12 HA-18 HA. In some embodiments, the difference between the hardness of the modified silicone rubber in the embodiments of the present disclosure and the hardness of the silicone rubber obtained by the polymerization of the structural unit shown in the general formula I-1 may be in a range of 14 HA-16 HA. For example, the difference between the hardness of the modified silicone rubber in the embodiments of the present disclosure and the hardness of the silicone rubber obtained by the polymerization of the structural unit shown in the general formula I-1 may be 0 HA, 1 HA, 10 HA, 13 HA, 15 HA, 16 HA, 25 HA, or 27 HA.

In the embodiments of the present disclosure, since the hardness of the silicone rubber obtained by different degrees of polymerization of the structural unit shown in the general formula I-1 is different, the comparison of the hardness of the modified silicone rubber in the embodiments of the present disclosure and the hardness of the silicone rubber obtained by the polymerization of the structural unit shown in the general formula I-1 may be performed under the condition that the modified silicone rubber in the embodiments of the present disclosure and the silicone rubber obtained by the polymerization of the structural unit shown in the general formula I-1 have the same silicone rubber structural units.

In some embodiments, when the silicone rubber component of the modified silicone rubber in the embodiments of the present disclosure is the RTV630 silicone rubber, a difference between the hardness of the modified silicone rubber and the hardness of the RTV630 silicone rubber (i.e., the silicone rubber obtained by the polymerization of the structural unit shown in the general formula I-1) may be in a range of 0-15 HA. In some embodiments, when the silicone rubber component of the modified silicone rubber is the RTV630 silicone rubber, the difference between the hardness of the modified silicone rubber and the hardness of the RTV630 silicone rubber may be in a range of 2 HA-13 HA. In some embodiments, when the silicone rubber component of the modified silicone rubber is the RTV630 silicone rubber, the difference between the hardness of the modified silicone rubber and the hardness of the RTV630 silicone rubber may be in a range of 4 HA-11 HA. In some embodiments, when the silicone rubber component of the modified silicone rubber is the RTV630 silicone rubber, the difference between the hardness of the modified silicone rubber and the hardness of the RTV630 silicone rubber may be in a range of 6 HA-9 HA. In some embodiments, when the silicone rubber component of the modified silicone rubber is the RTV630 silicone rubber, the difference between the hardness of the modified silicone rubber and the hardness of the RTV630 silicone rubber may be in a range of 7 HA-8 HA. For example, when the silicone rubber component of the modified silicone rubber is the RTV630 silicone rubber, the difference between the hardness of the modified silicone rubber and the hardness of the RTV630 silicone rubber may be 0 HA, 1 HA, 10 HA, or 15 HA.

In some embodiments, when the silicone rubber component of the modified silicone rubber in the embodiments of the present disclosure is the RTV615 silicone rubber, a difference between the hardness of the modified silicone rubber and the hardness of the RTV615 silicone rubber (i.e., the silicone rubber obtained by the polymerization of the structural unit shown in the general formula I-1) may be in a range of 0-30 HA. In some embodiments, when the silicone rubber component of the modified silicone rubber is the RTV615 silicone rubber, the difference between the hardness of the modified silicone rubber and the hardness of the RTV615 silicone rubber may be in a range of 2 HA-28 HA. In some embodiments, when the silicone rubber component of the modified silicone rubber is the RTV615 silicone rubber, the difference between the hardness of the modified silicone rubber and the hardness of the RTV615 silicone rubber may be in a range of 4 HA-26 HA. In some embodiments, when the silicone rubber component of the modified silicone rubber is the RTV615 silicone rubber, the difference between the hardness of the modified silicone rubber and the hardness of the RTV615 silicone rubber may be in a range of 6 HA-24 HA. In some embodiments, when the silicone rubber component of the modified silicone rubber is the RTV615 silicone rubber, the difference between the hardness of the modified silicone rubber and the hardness of the RTV615 silicone rubber may be in a range of 8 HA-22 HA. In some embodiments, when the silicone rubber component of the modified silicone rubber is the RTV615 silicone rubber, the difference between the hardness of the modified silicone rubber and the hardness of the RTV615 silicone rubber may be in a range of 10 HA-20 HA. In some embodiments, when the silicone rubber component of the modified silicone rubber is the RTV615 silicone rubber, the difference between the hardness of the modified silicone rubber and the hardness of the RTV615 silicone rubber may be in a range of 12 HA-18 HA. In some embodiments, when the silicone rubber component of the modified silicone rubber is the RTV615 silicone rubber, the difference between the hardness of the modified silicone rubber and the hardness of the RTV615 silicone rubber may be in a range of 14 HA-16 HA. For example, when the silicone rubber component of the modified silicone rubber is the RTV615 silicone rubber, the difference between the hardness of the modified silicone rubber and the hardness of the RTV615 silicone rubber may be 13 HA, 16 HA, 25 HA, or 27 HA.

In the embodiments of the present disclosure, the hardness of the modified silicone rubber may be in a range of 30 HA-70 HA. In some embodiments, the hardness of the modified silicone rubber in the embodiments of the present disclosure may be in a range of 34 HA-66 HA. In some embodiments, the hardness of the modified silicone rubber in the embodiments of the present disclosure may be in a range of 38 HA-62 HA. In some embodiments, the hardness of the modified silicone rubber in the embodiments of the present disclosure may be in a range of 42 HA-58 HA. In some embodiments, the hardness of the modified silicone rubber in the embodiments of the present disclosure may be in a range of 46 HA-54 HA. In some embodiments, the hardness of the modified silicone rubber in the embodiments of the present disclosure may be in a range of 48 HA-50 HA. For example, the hardness of the modified silicone rubber in the embodiments of the present disclosure may be 38 HA, 41 HA, 50 HA, 51 HA, 52 HA, 60 HA, or 65 HA.

In some embodiments, when the silicone rubber component of the modified silicone rubber in the embodiments of the present disclosure is the RTV630 silicone rubber, the hardness of the modified silicone rubber may be in a range of 45 HA-70 HA. In some embodiments, when the silicone rubber component of the modified silicone rubber in the embodiments of the present disclosure is the RTV630 silicone rubber, the hardness of the modified silicone rubber may be in a range of 50 HA-65 HA. In some embodiments, when the silicone rubber component of the modified silicone rubber in the embodiments of the present disclosure is the RTV630 silicone rubber, the hardness of the modified silicone rubber may be in a range of 55 HA-60 HA. For example, when the silicone rubber component of the modified silicone rubber in the embodiments of the present disclosure is the RTV630 silicone rubber, the hardness of the modified silicone rubber may be 50 HA, 51 HA, 60 HA, or 65 HA.

In some embodiments, when the silicone rubber component of the modified silicone rubber in the embodiments of the present disclosure is the RTV615 silicone rubber, the hardness of the modified silicone rubber may be in a range of 30 HA-60 HA. In some embodiments, when the silicone rubber component of the modified silicone rubber in the embodiments of the present disclosure is the RTV615 silicone rubber, the hardness of the modified silicone rubber may be in a range of 35 HA-55 HA. In some embodiments, when the silicone rubber component of the modified silicone rubber in the embodiments of the present disclosure is the RTV615 silicone rubber, the hardness of the modified silicone rubber may be in a range of 40 HA-50 HA. In some embodiments, when the silicone rubber component of the modified silicone rubber in the embodiments of the present disclosure is the RTV615 silicone rubber, the hardness of the modified silicone rubber may be in a range of 44 HA-46 HA. For example, when the silicone rubber component of the modified silicone rubber in the embodiments of the present disclosure is the RTV615 silicone rubber, the hardness of the modified silicone rubber may be 38 HA, 41 HA, 50 HA, or 52 HA.

In the embodiments of the present disclosure, the hardness of the modified silicone rubber may be obtained using a Shore durometer by referring to the GB/T531.1-2008 standard.

In the embodiments of the present disclosure, the structural unit shown in the general formula I-1 and the compound shown in the general formula I may have a structural correspondence. For example, the compound shown in the general formula I may react to obtain the structural unit shown in the I-1.

In the embodiments of the present disclosure, the mass fractions of the structural unit shown in the general formula I-1 and the compound shown in the general formula I may correspond.

In the embodiments of the present disclosure, the structural unit shown in the general formula II-1 and the compound shown in the general formula II may have a corresponding relationship in structure and mass fraction; the structural unit shown in the general formula III-1 and the compound shown in the general formula III may have a corresponding relationship in structure and mass fraction; the structural unit shown in the general formula VI and polycarbodiimide may have a corresponding relationship in structure and mass fraction.

In the embodiments of the present disclosure, according to mass fractions, the structural unit shown in the general formula I-1 may be 50-150 parts, and the structural unit shown in the general formula II-1 may be 25-150 parts.

In the embodiments of the present disclosure, according to mass fractions, the structural unit shown in the general formula I-1 may be 50-150 parts, and a sum of the structural unit shown in the general formula II-1 and the structural unit shown in the general formula III-1 may be 25-150 parts.

In the embodiments of the present disclosure, according to mass fractions, the structural unit shown in the general formula I-1 may be 50-150 parts, the structural unit shown in the general formula IV-1 may be 25-150 parts, and the structural unit shown in the general formula V or the general formula VI may be 0-20 (not 0) parts.

In the embodiments of the present disclosure, a viscosity of the modified silicone rubber may be greater than or equal to 10000 mpa·s. In the embodiments of the present disclosure, when the structural unit shown in the general formula I-1 can form the RTV615 silicone rubber, the viscosity of the modified silicone rubber may be in a range of 10000 mpa·s-30000 mpa·s. In the embodiments of the present disclosure, when the structural unit shown the general formula I-1 can form the RTV630 silicone rubber, the viscosity of the modified silicone rubber may be greater than 150000 mpa·s.

In the embodiments of the present disclosure, when the structural unit shown in the general formula II-1 and the structural unit shown in the general formula III-1 undergo an addition reaction, in the modified silicone rubber, a mass content of the structural unit shown in the general formula III-1 may be 15%-50%.

The embodiments of the present disclosure may further provide an application of the modified silicone rubber as a sound-transparent material. For example, the modified silicone rubber may be used as the sound-transparent material for a transducer. In some embodiments, the sound-transparent material may include a sound-transparent element applied to an ultrasound probe. In some embodiments, the sound-transparent material may be an acoustic lens material.

The embodiments of the present disclosure further provide an acoustic lens including the modified silicone rubber.

The embodiments of the present disclosure further provide an application of a modified material as the sound-transparent material. The embodiments of the present disclosure further provide a modified material of silicone rubber. In some embodiments, the modified material may be at least one of nitrile rubber, cis-polybutadiene, or fluoro rubber. In some embodiments, the sound-transparent material may be the acoustic lens material. In some embodiments, the nitrile rubber may be nitrile rubber as described above. In some embodiments, the cis-polybutadiene may be cis-polybutadiene as described above. In some embodiments, the fluoro rubber may be fluoro rubber as described above. In some embodiments, a raw material that can form the modified material may include a third component and a fourth component. More descriptions regarding the third component and the fourth component may be found elsewhere in the present disclosure, which is not repeated herein.

It should be noted that the above descriptions are for illustration and description purposes only, and does not limit the scope of the application of the present disclosure. Various modifications and variations may be made by those skilled in the art under the guidance of the present disclosure. However, such modifications and variations remain within the scope of the present disclosure.

In order to make the purposes, technical solutions, and advantages of the present disclosure more concise and clear, the present disclosure is described with the following specific examples, but the present disclosure is by no means limited to these examples. The embodiments described below are only preferred embodiments of the present disclosure, which can be used to describe the present disclosure, and should not be construed as limiting the scope of the present disclosure. It should be noted that any modifications, equivalent replacements and improvements made within the spirit and principles of the present disclosure shall be included within the protection scope of the present disclosure.

In order to better illustrate the present disclosure, the content of the present disclosure would be further described below in conjunction with the examples. The following are specific examples and contrast examples. The reagents and raw materials used in the embodiments of the present disclosure are all commercially available. Two-component RTV liquid silicone rubber RTV630 and RTV615 are produced by Momentive, and the first component and the second component are packaged separately. During use, the first component and the second component may be mixed well in a mass ratio of 10:1 (the first component/the second component=10:1), and then cured and molded at the room temperature or a high temperature (e.g., 50° C.-70° C.) to form the RTV630 silicone rubber and the RTV615 silicone rubber, respectively.

Raw materials of the fluoro rubber were purchased from Shanghai Silicon Mountain Polymer Material Co., Ltd., with a model of FAQ-008. The third component and the fourth component are packaged separately. During use, the third component and the fourth component may be mixed well in a mass ratio of 10:1 (the third component/the fourth component=10:1), and then cured and molded at the room temperature or a high temperature (e.g., 50° C.-70° C.).

Raw materials of the cis-polybutadiene were purchased from Yuyao Huihong Plastic Factory, with a model of PBR-4040. The third component and the fourth component are packaged separately. During use, the third component and the fourth component may be mixed well in a mass ratio of 10:1 (the third component/the fourth component=10:1), and then cured and molded at the room temperature or a high temperature (e.g., 50° C.-70° C.).

The raw materials of the nitrile rubber were purchased from Shandong Wang Brothers Plastic Technology Co., Ltd., with a model of LNBR820. The third component and the fourth component are packaged separately. During use, the third component and the fourth component may be mixed well in a mass ratio of 10:1 (the third component/the fourth component=10:1), and then cured and molded at the room temperature or a high temperature (e.g., 50° C.-70° C.).

An acoustic performance of the materials may be obtained by an oscilloscope through hydroacoustic measurement. A detection method and a calculation formula of the acoustic impedance and the sound attenuation may be as follows. A density of modified silicone rubber sample is calculated through a density formula: ρ=m/V, wherein m is a mass of a modified silicone rubber sample, and V is a volume of modified silicone rubber sample, and then, a material sound velocity and a sound attenuation coefficient of the modified silicone rubber sample may be obtained by a water insertion method, and the acoustic impedance of the modified silicone rubber sample may be calculated according to a calculation formula as follows:

C=(I₁−I₂)C_{0/}(ΔtC₀+(I₁−I₂)),

α=(20 Ig(A₁/A₂))/(I₁−I₂)+α₀,

Z=ρC,

where C represents the material sound velocity of the modified silicone rubber, I₁ is a thickness of modified silicone rubber sample 1, I₂ is a thickness of modified silicone rubber sample 2, Δt is a time difference of sound propagation caused by the insertion of the modified silicone rubber sample 1 and the modified silicone rubber sample 2, Co is a sound velocity in water, α₀ is a sound attenuation coefficient in water, A₁ and A₂ are an amplitude of pulse signals received by modified silicone rubber sample 1 and modified silicone rubber sample 2, respectively, α is a sound attenuation coefficient of the modified silicone rubber sample in water, and Z is the acoustic impedance of the modified silicone rubber sample.

Mechanical properties (e.g., hardness) of the materials may be obtained using a Shore durometer by referring to the GB/T531.1-2008 standard.

Contrast Example 1: 100 g of the first component of the RTV630 silicone rubber and 10 g of the second component of the RTV630 silicone rubber were weighed and placed in a beaker. The mixture is mixed evenly with a stirring device and defoamed with a defoaming device, then poured into a mold and cured in a constant temperature and humidity box at 25° C. and 50% RH (relative humidity) for 48 h, and then molded to obtain RTV630 silicone rubber with an acoustic impedance of 1.30 MRayl, a sound attenuation of 30.0 dB/cm at 5 MHz, and a Shore hardness (Shore A) of 50. The RTV630 silicone rubber contains Si—O bonds, methyl, vinyl, and white carbon black.

Example 1: 100 g of the first component of the RTV630 silicone rubber and 50 g of the third component of the fluoro rubber were weighed and mixed evenly with a stirring device, then 10 g of the second component of the RTV630 silicone rubber and 5 g of the fourth component of the fluoro rubber were added. The mixture is mixed evenly with a stirring device and defoamed with a defoaming device, then poured into a mold and cured in a constant temperature and humidity box at 25° C. and 50% RH for 48 h, and then molded to obtain RTV630 silicone rubber/fluoro rubber composite material (i.e., the modified silicone rubber) with an acoustic impedance of 1.50 MRayl, a sound attenuation of 31.3 dB/cm at 5 MHz, and a Shore hardness (Shore A) of 65. The obtained RTV630 silicone rubber/fluoro rubber composite material contains Si—O bonds, methyl, vinyl, white carbon black, and fluorine atoms.

By comparing the Example 1 with the Contrast Example 1, it can be seen that the addition of the liquid fluoro rubber to the RTV630 silicone rubber effectively improves the impedance and the hardness of the material, and the sound attenuation of the material increases very little.

Example 2: 100 g of the first component of the RTV630 silicone rubber and 50 g of the third component of the cis-polybutadiene were weighed and mixed evenly, then 10 g of the second component of the RTV630 silicone rubber and 5 g of the fourth component of the cis-polybutadiene were added. The mixture is mixed evenly with a stirring device and defoamed with a defoaming device, then poured into a mold and cured in a constant temperature and humidity box at 25° C. and 50% RH for 48 h, and then molded to obtain RTV630 silicone rubber/cis-polybutadiene composite material (i.e., the modified silicone rubber) with an acoustic impedance of 1.42 MRayl, a sound attenuation of 41.0 dB/cm at 5 MHz, and a Shore hardness (Shore A) of 60. The obtained RTV630 silicone rubber/cis-polybutadiene composite material contains Si—O bonds, methyl, vinyl, white carbon black, and butadienyl.

By comparing the Example 2 with the Contrast Example 1, it can be seen that the addition of the liquid cis-polybutadiene to the RTV630 silicone rubber effectively improves the impedance and the hardness of the material, and slightly improves the sound attenuation of the material.

Example 3: 100 g of the first component of the RTV630 silicone rubber and 50 g of the third component of the nitrile rubber were weighed and mixed evenly with a stirring device, then 10 g of the second component of the RTV630 silicone rubber and 5 g of the fourth component of the nitrile rubber were added. The mixture is mixed evenly with a stirring device and defoamed with a defoaming device, then poured into a mold and cured in a constant temperature and humidity box at 25° C. and 50% RH for 48 h, and then molded to obtain an RTV630 silicone rubber/nitrile rubber composite material (i.e., the modified silicone rubber) with an acoustic impedance of 1.39 MRayl, a sound attenuation of 22.0 dB/cm at 5 MHz, and a Shore hardness (Shore A) of 50. The obtained RTV630 silicone rubber/nitrile rubber composite material contains Si—O bonds, methyl, vinyl, acrylonitrile groups, white carbon black, and butadienyl.

By comparing the Example 3 with the Contrast Example 1, it can be seen that the addition of the liquid nitrile rubber to the RTV630 silicone rubber effectively improves the impedance of the material and reduces the sound attenuation of the material without changing the hardness.

Example 4: 100 g of the first component of the RTV630 silicone rubber and 100 g of the third component of nitrile rubber were weighed and mixed evenly with a stirring device, then 10 g of the second component of the RTV630 silicone rubber and 10 g of the fourth component of the nitrile rubber were added. The mixture is mixed evenly with a stirring device and defoamed with a defoaming device, then poured into a mold and cured in a constant temperature and humidity box at 25° C. and 50% RH for 48 h, and then molded to obtain an RTV630 silicone rubber/nitrile rubber composite material (i.e., the modified silicone rubber) with an acoustic impedance of 1.36 MRayl, a sound attenuation of 20.0 dB/cm at 5 MHz, and a Shore hardness (Shore A) of 51. The obtained RTV630 silicone rubber/nitrile rubber composite material contains Si—O bonds, methyl, vinyl, acrylonitrile, white carbon black, and butadienyl.

By comparing the Example 4 with the Example 3 and the Contrast Example 1, it can be seen that the increasing of the amount of the liquid nitrile rubber in the RTV630 silicone rubber reduces the sound attenuation of the material, but reduces the impedance, and causes a small change in the hardness.

Contrast Example 2: 100 g of the first component of the RTV615 silicone rubber and 10 g of the second component of RTV615 silicone rubber were weighed and placed in a beaker. The mixture is mixed evenly with a stirring device and defoamed with a defoaming device, then poured into a mold and cured in a constant temperature and humidity box at 25° C. and 50% RH for 48 h, and then molded to obtain RTV615 silicone rubber with an acoustic impedance of 1.05 MRayl, a sound attenuation of 15.4 dB/cm at 5 MHz, and a Shore hardness (Shore A) of 25. The RTV615 silicone rubber contains Si—O bonds, methyl, and vinyl.

Example 5: 100 g of the first component of the RTV615 silicone rubber and 50 g of the second component of the fluoro rubber were weighed and mixed evenly with a stirring device, then 10 g of the second component of the RTV615 silicone rubber and 5 g of the fourth component of the fluoro rubber were added. The mixture is mixed evenly with a stirring device and defoamed with a defoaming device, then poured into a mold and cured in a constant temperature and humidity box at 25° C. and 50% RH for 48 h, and then molded to obtain an RTV615 silicone rubber/fluoro rubber composite material (i.e., the modified silicone rubber) with an acoustic impedance of 1.31 MRayl, a sound attenuation of 25.0 dB/cm at 5 MHz, and a Shore hardness (Shore A) of 50. The RTV615 silicone rubber/fluoro rubber composite contains Si—O bonds, methyl, vinyl, and fluorine atoms.

By comparing the Example 5 with the Contrast Example 2, it can be seen that the addition of the liquid fluoro rubber to the RTV615 silicone rubber improves the impedance and the hardness of the material, but sharply increases the sound attenuation.

Example 6: 100 g of the first component of the RTV615 silicone rubber and 50 g of the third component of the cis-polybutadiene were weighed and mixed evenly, then 10 g of the second component of the RTV615 silicone rubber and 5 g of the fourth component of the cis-polybutadiene were added. The mixture is mixed evenly with a stirring device and defoamed with a defoaming device, then poured into a mold and cured in a constant temperature and humidity box at 25° C. and 50% RH for 48 h, and then molded to obtain an RTV615 silicone rubber/cis-polybutadiene composite material (i.e., the modified silicone rubber) with an acoustic impedance of 1.36 MRayl, a sound attenuation of 29.1 dB/cm at 5 MHz, and a Shore hardness (Shore A) of 52. The RTV615 silicone rubber/cis-polybutadiene compound contains Si—O bonds, methyl, vinyl, and butadienyl.

By comparing the Example 6 with the Contrast Example 2, it can be seen that the addition of the liquid cis-polybutadiene to the RTV615 silicone rubber improves the impedance and the hardness of the material, but sharply increases the sound attenuation.

Example 7: 100 g of the first component of the RTV615 silicone rubber and 50 g of the third component of the nitrile rubber were weighed and mixed evenly with a stirring device, then 10 g of the second component of the RTV615 silicone rubber and 5 g of the fourth component of the nitrile rubber were added. The mixture is mixed evenly with a stirring device and defoamed with a defoaming device, then poured into a mold and cured in a constant temperature and humidity box at 25° C. and 50% RH for 48 h, and then molded to obtain an RTV615 silicone rubber/nitrile rubber composite material (i.e., the modified silicone rubber) with an acoustic impedance of 1.26 MRayl, a sound attenuation of 13.1 dB/cm at 5 MHz, and a Shore hardness (Shore A) of 38. The obtained RTV615 silicone rubber/nitrile rubber composite material contains Si-O bonds, methyl, vinyl, acrylonitrile group, and butadienyl.

By comparing the Example 7 with the Contrast Example 2, it can be seen that the addition of the liquid nitrile rubber to the RTV615 silicone rubber improves the impedance and the hardness of the material, and reduces the sound attenuation of the material.

Example 8: 100 g of the first component of the RTV615 silicone rubber and 100 g of the third component of the nitrile rubber were weighed and mixed evenly with a stirring device, then 10 g of the second component of the RTV615 silicone rubber and 10 g of the fourth component of the nitrile rubber were added. The mixture is mixed evenly with a stirring device and defoamed with a defoaming device, then poured into a mold and cured in a constant temperature and humidity box at 25° C. and 50% RH for 48 h, and then molded to obtain an RTV615 silicone rubber/nitrile rubber composite material (i.e., the modified silicone rubber) with an acoustic impedance of 1.37 MRayl, a sound attenuation of 12.5 dB/cm at 5 MHz, and a Shore hardness (Shore A) of 41. The obtained RTV615 silicone rubber/nitrile rubber composite material contains Si—O bonds, methyl, vinyl, acrylonitrile groups, and butadiene.

By comparing the Example 8 with the Example 7 and the Contrast Example 2, it can be seen that the increasing of the amount of the liquid nitrile rubber in the RTV615 silicone rubber reduces the sound attenuation of the material and increases the impedance and the hardness of the material.

Example 9: 100 g of the third component of the nitrile rubber and 10 g of the fourth component of the nitrile rubber were weighed and placed in a beaker. The mixture is mixed evenly with a stirring device and defoamed with a defoaming device, then poured into a mold and cured in a constant temperature and humidity box at 25° C. and 50% RH for 48 h, and then molded to obtain a two-component nitrile rubber with an acoustic impedance of 1.52 MRayl, a sound attenuation of 10.5 dB/cm at 5 MHz, and a Shore hardness (Shore A) of 60. The obtained two-component nitrile rubber contains butadienyl and acrylonitrile groups.

Example 10: 100 g of the third component of the cis-polybutadiene and 10 g of the fourth component of the cis-polybutadiene were weighed and placed in a beaker. The mixture is mixed evenly with a stirring device and defoamed with a defoaming device, then poured into a mold and cured in a constant temperature and humidity box at 25° C. and 50% RH for 48 h, and then molded to obtain a two-component cis-polybutadiene with an acoustic impedance of 1.61 MRayl, a sound attenuation of 40.0 dB/cm (5 MHz), and a Shore hardness (Shore A) of 80. The obtained two-component cis-polybutadiene contains butadienyl.

Example 11: 100 g of the third component of the fluoro rubber and 10 g of the fourth component of the fluoro rubber were weighed and placed in a beaker. The mixture is mixed evenly with a stirring device and defoamed with a defoaming device, then poured into a mold and cured in a constant temperature and humidity box at 25° C. and 50% RH for 48 h, and then molded to obtain a two-component fluoro rubber with an acoustic impedance of 1.70 MRayl, a sound attenuation of 40.0 dB/cm (5 MHz), and a Shore hardness (Shore A) of 60. The obtained two-component fluoro rubber contains fluorine atoms.

The raw materials and experimental results of the Examples 1-11 and the Contrast Examples 1-2 may be shown in Table 2 and Table 3, respectively. It can be seen from the Examples 1-11 and the Contrast Examples 1 and 2 that the improvement of the acoustic performance (e.g., the acoustic impedance, the sound attenuation) and the hardness of the RTV630 silicone rubber and the RTV615 silicone rubber comes from the good acoustic performance and the hardness of the rubber (e.g., the fluoro rubber, the cis-polybutadiene, the nitrile rubber) blended with the RTV630 silicone rubber and the RTV615 silicone rubber.

TABLE 2
Raw materials of the Examples 1-11 and the Contrast Example 1-2

| | | Monomer of silicon rubber | | | | | Monomer of modified material | | | |
|---|---|---|---|---|---|---|---|---|---|---|
| | | | | | Type | | | Type of | | Type of |
| | | Amount | Type of first | Amount | of second | | Amount | third | Amount | fourth |
| | | of first | component | of second | component | | of third | component | of fourth | component |
| | Silicon | compo- | (main | compo- | (main | modified | compo- | (main | compo- | (main |
| No. | rubber | nent/g | component) | nent/g | component) | material | nent/g | component) | nent/g | component) |
| Contrast | RTV630 | 100 | Polyvinyl- | 10 | Platinum | / | / | / | / | / |
| Exam- | | | polysiloxane + | | catalyst | | | | | |
| ple 1 | | | Polymethyl- | | | | | | | |
| | | | hydrosiloxane + | | | | | | | |
| | | | White carbon | | | | | | | |
| | | | black | | | | | | | |
| Exam- | RTV630 | 100 | Polyvinyl- | 10 | Platinum | Fluoro | 50 | Fluorine- | 5 | Polycarbo- |
| ple 1 | | | polysiloxane + | | catalyst | rubber | | containing | | diimide |
| | | | Polymethyl- | | | | | carbon | | |
| | | | hydrosiloxane + | | | | | chain | | |
| | | | White carbon | | | | | | | |
| | | | black | | | | | | | |
| Exam- | RTV630 | 100 | Polyvinyl- | 10 | Platinum | Cis- | 50 | Butadiene | 5 | Organotin |
| ple 2 | | | polysiloxane + | | catalyst | polybutadiene | | | | |
| | | | Polymethyl- | | | | | | | |
| | | | hydrosiloxane + | | | | | | | |
| | | | White carbon | | | | | | | |
| | | | black | | | | | | | |
| Exam- | RTV630 | 100 | Polyvinyl- | 10 | Platinum | Nitrile | 50 | Butadiene + | 5 | Organotin |
| ple 3 | | | polysiloxane + | | catalyst | rubber | | Acrylonitrile | | |
| | | | Polymethyl- | | | | | | | |
| | | | hydrosiloxane + | | | | | | | |
| | | | White carbon | | | | | | | |
| | | | black | | | | | | | |
| Exam- | RTV630 | 100 | Polyvinyl- | 10 | Platinum | Nitrile | 100 | Butadiene + | 10 | Organotin |
| ple 4 | | | polysiloxane + | | catalyst | rubber | | Acrylonitrile | | |
| | | | Polymethyl- | | | | | | | |
| | | | hydrosiloxane + | | | | | | | |
| | | | White carbon | | | | | | | |
| | | | black | | | | | | | |
| Contrast | RTV615 | 100 | Polyvinyl- | 10 | Platinum | / | / | / | / | / |
| Exam- | | | polysiloxane + | | catalyst | | | | | |
| ple 2 | | | Polymethyl- | | | | | | | |
| | | | hydrosiloxane | | | | | | | |
| Exam- | RTV615 | 100 | Polyvinyl- | 10 | Platinum | Fluoro | 50 | Fluorine- | 5 | Polycarbo- |
| ple 5 | | | polysiloxane + | | catalyst | rubber | | containing | | diimide |
| | | | Polymethyl- | | | | | carbon | | |
| | | | hydrosiloxane | | | | | chain | | |
| Exam- | RTV615 | 100 | Polyvinyl- | 10 | Platinum | Cis- | 50 | Butadiene | 5 | Organotin |
| ple 6 | | | polysiloxane + | | catalyst | polybutadiene | | | | |
| | | | Polymethyl- | | | | | | | |
| | | | hydrosiloxane | | | | | | | |
| Exam- | RTV615 | 100 | Polyvinyl- | 10 | Platinum | Nitrile | 50 | Butadiene + | 5 | Organotin |
| ple 7 | | | polysiloxane + | | catalyst | rubber | | Acrylonitrile | | |
| | | | Polymethyl- | | | | | | | |
| | | | hydrosiloxane | | | | | | | |
| Exam- | RTV615 | 100 | Polyvinyl- | 10 | Platinum | Nitrile | 100 | Butadiene + | 10 | Organotin |
| ple 8 | | | polysiloxane + | | catalyst | rubber | | Acrylonitrile | | |
| | | | Polymethyl- | | | | | | | |
| | | | hydrosiloxane | | | | | | | |
| Exam- | / | / | / | / | / | Nitrile | 100 | Butadiene + | 10 | Organotin |
| ple 9 | | | | | | rubber | | Acrylonitrile | | |
| Exam- | / | / | / | / | / | Cis- | 100 | Butadiene | 10 | Organotin |
| ple 10 | | | | | | polybutadiene | | | | |
| Exam- | / | / | / | / | / | Fluoro | 100 | Fluorine- | 10 | Polycarbo- |
| ple 11 | | | | | | rubber | | containing | | diimide |
| | | | | | | | | carbon | | |
| | | | | | | | | chain | | |

A unit of the amount in Table 2 is g, and “/” means that this component is not contained. When the first component contains a plurality of components, the amount of the first component refers to a sum of masses of the plurality of components; when the third component contains a plurality of components, the amount of the third component refers to a sum of masses of the plurality of components. For example, the third component in the Example 3 contains the butadiene and the acrylonitrile, the amount of the third component refers to the sum of the masses of the butadiene and the acrylonitrile, i.e.,100 g, and a ratio of the butadiene to the acrylonitrile may be a conventional ratio in this field.

In Table 2, (1) the structural formula of the polyvinylpolysiloxane is:

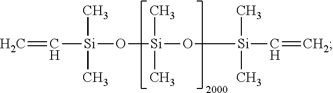

(2) The structural formula of the butadiene is:

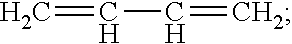

(3) The structural formula of the acrylonitrile is:

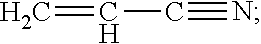

(4) The structural formula of the fluorine-containing carbon chain is:

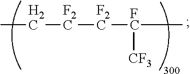

(5) The structural formula of the polycarbodiimide is HN═C═NH; (6) The platinum catalyst is a homogeneous platinum catalyst; (7) Organotin is dibutyltin dilaurate or stannous octoate.

TABLE 3
Experimental results of the Examples 1-11
and the Contrast Example 1-2

| | | | | Sound | Shore |
|---|---|---|---|---|---|
| | Silicone | Modified | Acoustic | attenuation | hard- |
| No. | rubber | material | impedance | at 5 MHz | ness |
| Contrast | RTV630 | / | 1.30 MRayl | 30.0 dB/cm | 50 |
| Example 1 | | | | | |
| Example 1 | RTV630 | Fluoro rubber | 1.50 MRayl | 31.3 dB/cm | 65 |
| Example 2 | RTV630 | Cis- | 1.42 MRayl | 41.0 dB/cm | 60 |
| | | polybutadiene | | | |
| Example 3 | RTV630 | Nitrile rubber | 1.39 MRayl | 22.0 dB/cm | 50 |
| Example 4 | RTV630 | Nitrile rubber | 1.36 MRayl | 20.0 dB/cm | 51 |
| Contrast | RTV615 | / | 1.05 MRayl | 15.4 dB/cm | 25 |
| Example 2 | | | | | |
| Example 5 | RTV615 | Fluoro rubber | 1.31 MRayl | 25.0 dB/cm | 50 |
| Example 6 | RTV615 | Cis- | 1.36 MRayl | 29.1 dB/cm | 52 |
| | | polybutadiene | | | |
| Example 7 | RTV615 | Nitrile rubber | 1.26 MRayl | 13.1 dB/cm | 38 |
| Example 8 | RTV615 | Nitrile rubber | 1.37 MRayl | 12.5 dB/cm | 41 |
| Example 9 | / | Nitrile rubber | 1.52 Mrayl | 10.5 dB/cm | 60 |
| Example 10 | / | Cis- | 1.61 MRayl | 40.0 dB/cm | 80 |
| | | polybutadiene | | | |
| Example 11 | / | Fluoro rubber | 1.70 MRayl | 40.0 dB/cm | 60 |

The main structural units of the final materials prepared in the Examples 1-11 and the Contrast Examples 1-2 may be shown in Table 4.

TABLE 4
Main structural units of the final materials prepared in the Examples 1-11 and
the Contrast Example 1-2

| | Silicone | Modified | |
|---|---|---|---|
| No. | rubber | material | Main structural unit of final material |
| Contrast Example 1 | RTV630 | / | 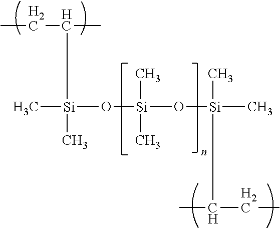 |
| Example 1 | RTV630 | Fluoro rubber | 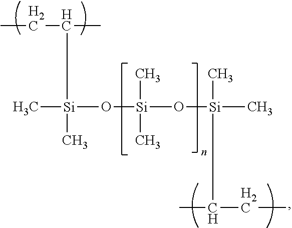 |
| | | | 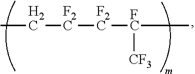 |
| | | | 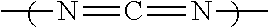 |
| Example 2 | RTV630 | Cis- polybutadiene | 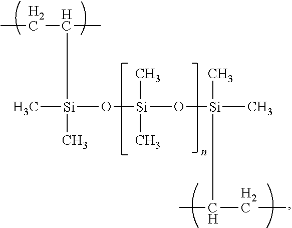 |
| | | | 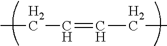 |
| Example 3 | RTV630 | Nitrile rubber | 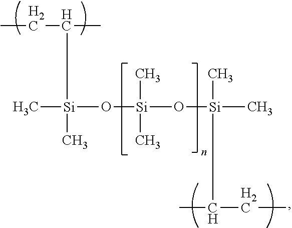 |
| | | | 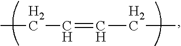 |
| | | | 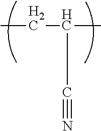 |
| Example 4 | RTV630 | Nitrile rubber | 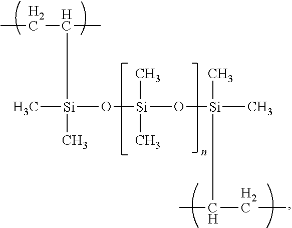 |
| | | | 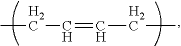 |
| | | | 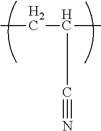 |
| Contrast Example 2 | RTV615 | / | 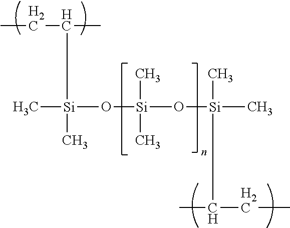 |
| Example 5 | RTV615 | Fluoro rubber | 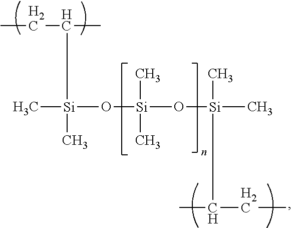 |
| | | | 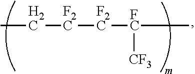 |
| | | | 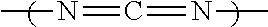 |
| Example 6 | RTV615 | Cis- polybutadiene | 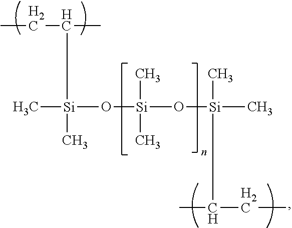 |
| | | | 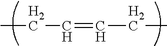 |
| Example 7 | RTV615 | Nitrile rubber | 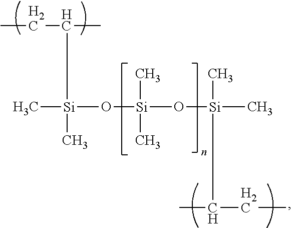 |
| | | | 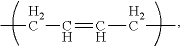 |
| | | | 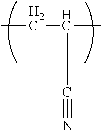 |
| Example 8 | RTV615 | Nitrile rubber | 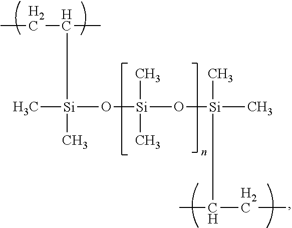 |
| | | | 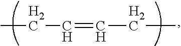 |
| | | | 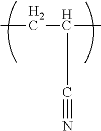 |
| Example 9 | / | Nitrile rubber | 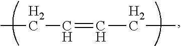 |
| | | | 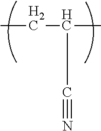 |
| Example 10 | / | Cis- polybutadiene | 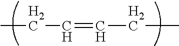 |
| Example 11 | / | Fluoro rubber | 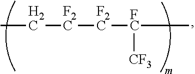 |
| | | | 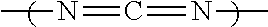 |

A viscosity of the modified silicone rubber of the RTV615 silicone rubber obtained in the Examples 5-8 may be in a range of 10000 mpa·s-30000 mpa·s. The viscosity of the modified silicone rubber of the RTV630 silicone rubber obtained in the Examples 1-4 may be greater than or equal to 150000 mpa·s.

The possible beneficial effects of the embodiments of the present disclosure may include, but are not limited to the following. (1) A blending modification method of the silicone rubber is proposed, which can effectively improve the acoustic impedance of the modified silicone rubber, reduce the sound attenuation of the modified silicone rubber, and improve the mechanical strength of the modified silicone rubber. For example, the Shore hardness of the RTV630 silicone rubber can be increased from 50 to 51-65, the acoustic impedance can be increased from 1.30 MRayl to 1.36 MRayl-1.50 MRayl, and the sound attenuation at 5 MHz can be reduced from 30 dB/cm to 20.0 dB/cm. As another example, the Shore hardness of the RTV615 silicone rubber can be increased from 25 to 38-52, the acoustic impedance can be increased from 1.05 MRayl to 1.26 MRayl-1.37 MRayl, and the sound attenuation can be reduced from 15.4 dB/cm to 12.5 dB/cm-13.1 dB/ cm. (2) The prepared modified silicone rubber has an acoustic impedance matching with the human body, and can be used as the acoustic lens of the ultrasonic probe, which not only has a long service life, but also improves the imaging sensitivity and the imaging quality of the ultrasonic diagnostic equipment. (3) The preparation process of the modified silicone rubber is simple, and the modified silicone rubber can be molded at the room temperature, which satisfies the preparation requirements of conventional acoustic lenses.

The basic concept has been described above. Obviously, for those skilled in the art, the above detailed disclosure is only an example, and does not constitute a limitation to the present disclosure. Although not expressly stated here, those skilled in the art may make various modifications, improvements and corrections to the present disclosure. Such modifications, improvements and corrections are suggested in this disclosure, so such modifications, improvements and corrections still belong to the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This method of disclosure does not, however, imply that the subject matter of the disclosure requires more features than are recited in the claims. Rather, claimed subject matter may lie in less than all features of a single foregoing disclosed embodiment.

In some embodiments, counts describing the quantity of components and attributes are used. It should be understood that such counts used in the description of the embodiments use the modifiers “about”, “approximately” or “substantially” in some examples. Unless otherwise stated, “about”, “approximately” or “substantially” indicates that the stated figure allows for a variation of ±20%. Accordingly, in some embodiments, the numerical parameters used in the disclosure and claims are approximations that can vary depending upon the desired characteristics of individual embodiments. In some embodiments, numerical parameters should consider the specified significant digits and adopt the general digit retention method. Although the numerical ranges and parameters used in some embodiments of the present disclosure to confirm the breadth of the range are approximations, in specific embodiments, such numerical values are set as precisely as practicable.

Each of the patents, patent applications, publications of patent applications, and other material, such as articles, books, specifications, publications, documents, things, and/or the like, referenced herein is hereby incorporated herein by this reference in its entirety for all purposes, Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail?

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. Modified silicone rubber prepared by a composition of raw materials, wherein the composition of raw materials includes:
a raw material that can form silicone rubber and includes a first component and a second component; and
a raw material that can form a modified material and includes a third component and a fourth component.

2. The modified silicone rubber of claim 1, wherein
a mass fraction of the first component is in a range of 50-150;
a mass fraction of the second component is in a range of 0-20 and greater than 0;
a mass fraction of the third component is in a range of 25-150; or
a mass fraction of the fourth component is in a range of 0-20 and greater than 0.

3. The modified silicone rubber of claim 1, wherein
a mass ratio of the first component, the second component, the third component, and the fourth component is 10:1:(5-10):(0.5-1).

4. The composition modified silicone rubber of claim 1, wherein
the first component includes vinyl silicone rubber and a cross-linking agent; or
the second component includes a catalyst capable of catalyzing an addition reaction of the vinyl silicone rubber and the cross-linking agent.

5. The modified silicone rubber of claim 4, wherein
a structural formula of the vinyl silicone rubber is shown in formula I: 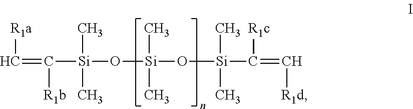
where R₁a, R₁b, R₁c, and R₁d are independently selected from H, substituted or unsubstituted C₁-C₅ straight chain or branched chain alkyl, substituted or unsubstituted C₆-C₂₀ aryl, n≥1000:
the cross-linking agent includes a Si—H bond; or
the second component includes at least one of transition metals of Group VIII of a periodic table, a compound thereof, or a complex thereof.

6. The modified silicone rubber of claim 4, wherein the second component further includes at least one of methyl silicone oil, vinyl silicone oil, hydroxyl silicone oil, hydroxymethyl fluoro silicone oil, or epoxy-terminated silicone oil.

7. The modified silicone rubber of claim 5, wherein a structural formula of the vinyl silicone rubber is shown in formula I′:

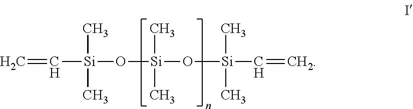

8. The modified silicone rubber of claim 5, wherein the cross-linking agent is polymethylhydrogensiloxane.

9. The modified silicone rubber of claim 4, wherein the first component further includes at least one of an inhibitor or a filler,

10. The modified silicone rubber of claim 9, wherein the inhibitor includes at least one of an alkynol compound, a nitrogen-containing compound, or organic peroxide.

11. The modified silicone rubber of claim 9, wherein the filler includes at least one of white carbon black, titanium dioxide, quartz powder, aluminum oxide, zinc oxide, or tungsten oxide.

12. The modified silicone rubber of claim 1, wherein:
the third component includes a butadiene compound and an acrylonitrile compound, and the fourth component includes a catalyst capable of catalyzing an addition reaction of the butadiene compound and the acrylonitrile compound; or
the third component includes the butadiene compound, and the fourth component includes a catalyst capable of catalyzing an addition reaction of the butadiene compound; or
the third component includes a fluorine-containing carbon chain, and the fourth component includes polycarbodiimide.

13. The modified silicone rubber of claim 12, wherein,
a structural formula of the butadiene compound is shown in formula II, 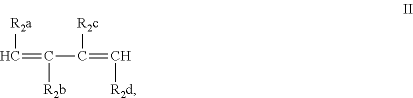
where R₂a, R₂b, R₂c, and R₂d are independently selected from H, substituted or unsubstituted C₁-C₃ straight chain or branched chain alkyl, substituted or unsubstituted C₆-C₂₀ aryl;
a structural formula of the acrylonitrile compound is shown in formula III, 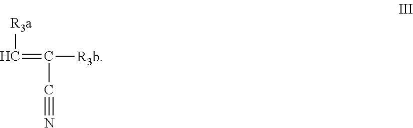
where R₃a and R₃b are independently selected from H, substituted or unsubstituted C₁-C₃ straight chain or branched chain alkyl, substituted or unsubstituted C₆-C₂₀ aryl;
a structural formula of the fluorine-containing carbon chain is shown in formula IV, 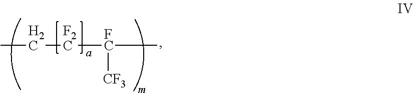
where m is not less than 200, a is in a range of 1-8, and a is an integer; or
the fourth component includes at least one of transition metals of Group VIII of the periodic table, a compound thereof, or a complex thereof.

14. The modified silicone rubber of claim 13, wherein the fourth component includes platinum, a platinum-containing compound, a platinum-containing complex, organotin, or organobismuth.

15. (canceled)

16. The modified silicone rubber of claim 13, wherein,
the butadiene compound is 1,3-butadiene, or
the acrylonitrile compound is acrylonitrile.

17. A preparation method of modified silicone rubber, comprising:
mixing a composition of raw materials for preparing the modified silicone rubber, and
obtaining the modified silicone rubber by curing and molding the mixture, wherein the composition of raw materials for preparing the modified silicone rubber includes:
a raw material that can form silicone rubber and includes a first component and a second component; and
a raw material that can form a modified material and includes a third component and a fourth component.

18. The modified silicone rubber of claim 1, wherein,
an acoustic impedance of the modified silicone rubber is within a range of 1.25 Mrayl-1.50 Mrayl;
a sound attenuation coefficient of the modified silicone rubber at a frequency of 5 MHz is not greater than 42 dB/cm; or
a Shore hardness of the modified silicone rubber is in a range of 30 HA-70 HA.

19. The modified silicone rubber of claim 1, wherein the modified silicone rubber is used as a sound-transparent material.

20. The modified silicone rubber of claim 19, wherein the sound-transparent material includes a sound-transparent element applied to an ultrasonic probe.

21-26. (canceled)

27. An ultrasonic probe including a sound-transparent element, the sound-transparent element including modified silicone rubber prepared by a composition of raw materials, wherein the composition of raw materials includes:
a raw material that can form silicone rubber and includes a first component and a second component; and
a raw material that can form a modified material and includes a third component and a fourth component.
